SEQUENCE LISTING (51 sequences)

SEQ ID NO: 1 - DNA, 19 bp, Artificial Sequence
  The same as those in human.
tcagttacggaaacgatgc

SEQ ID NO: 2 - DNA, 19 bp, Artificial Sequence
  The same as those in human.
gattatgcggatcaaacct

SEQ ID NO: 3 - DNA, 19 bp, Artificial Sequence
  The same as those in human.
cgggacccggtcgccagga

SEQ ID NO: 4 - DNA, 19 bp, Artificial Sequence
  The same as those in human.
atccgcacggataagaacg

SEQ ID NO: 5 - DNA, 19 bp, Artificial Sequence
  The same as those in human.
tgcgacccggacgacgaga

SEQ ID NO: 6 - DNA, 19 bp, Artificial Sequence
  The same as those in human.
ccagcttcggaacaagaga

SEQ ID NO: 7 - DNA, 19 bp, Artificial Sequence
  The same as those in human.
tgaacaacggattgagcta

SEQ ID NO: 8 - DNA, 19 bp, Artificial Sequence
  The same as those in human.
agaggaacggagcgagtcc

SEQ ID NO: 9 - DNA, 19 bp, Artificial Sequence
  The same as those in human.
atgtcaccggagttgtgcg

SEQ ID NO: 10 - DNA, 19 bp, Artificial Sequence
  The same as those in human.
gactcgccgggccctattc

SEQ ID NO: 11 - DNA, 19 bp, Artificial Sequence
  The same as those in human.
atgtccacggaagaggaga

SEQ ID NO: 12 - DNA, 19 bp, Artificial Sequence
  The same as those in human.
aagatcccggacgcacaga

SEQ ID NO: 13 - DNA, 19 bp, Artificial Sequence
  The same as those in human.
ccttcagcggccagtagca

SEQ ID NO: 14 - DNA, 19 bp, Artificial Sequence
  The same as those in human.
aaagctccgggtcttaggc

SEQ ID NO: 15 - DNA, 19 bp, Artificial Sequence
  The same as those in human.
gagtctccggggctctatg

SEQ ID NO: 16 - DNA, 19 bp, Artificial Sequence
  The same as those in human.
tgccccccggagccgcgag

SEQ ID NO: 17 - DNA, 19 bp, Artificial Sequence
  The same as those in human.
gaggctgcggattgtgcga

SEQ ID NO: 18 - DNA, 19 bp, Artificial Sequence
  The same as those in human.
ctttctacggacgtgggat

SEQ ID NO: 19 - DNA, 19 bp, Artificial Sequence
  The same as those in human.
tttctgccggagagctttg

SEQ ID NO: 20 - DNA, 19 bp, Artificial Sequence
  The same as those in human.
aagattccgggagttggtg

SEQ ID NO: 21 - DNA, 19 bp, Artificial Sequence
  The same as those in human.
gccggcccggattgacgag

SEQ ID NO: 22 - DNA, 19 bp, Artificial Sequence
  The same as those in human.
aaggggtcggtggaccggt

SEQ ID NO: 23 - DNA, 19 bp, Artificial Sequence
  The same as those in human.
ggtggaccggtcgatgtat

SEQ ID NO: 24 - DNA, 19 bp, Artificial Sequence
  The same as those in human.
ctgtgcacggaactgaaca

SEQ ID NO: 25 - DNA, 19 bp, Artificial Sequence
  The same as those in human.
gtgcctgcggtgccagaaa

SEQ ID NO: 26 - DNA, 19 bp, Artificial Sequence
  The same as those in human.
gcaagttcggcagcagctt

SEQ ID NO: 27 - DNA, 19 bp, Artificial Sequence
  The same as those in human.
atagttgcggagagtctgc

SEQ ID NO: 28 - DNA, 19 bp, Artificial Sequence
  The same as those in human.
tgaatttcggcacctgcaa

SEQ ID NO: 29 - DNA, 19 bp, Artificial Sequence
  The same as those in human.
tcccagaacggaggcgaac

SEQ ID NO: 30 - DNA, 19 bp, Artificial Sequence
  The same as those in human.
tacattccggaaagattgt

SEQ ID NO: 31 - DNA, 19 bp, Artificial Sequence
  The same as those in human.
gttattttggttcgagaga

SEQ ID NO: 32 - DNA, 19 bp, Artificial Sequence
  The same as those in human.
taatgggggcgagctgttt

SEQ ID NO: 33 - DNA, 19 bp, Artificial Sequence
  The same as those in human.
tggaccccggattgctgct

SEQ ID NO: 34 - DNA, 19 bp, Artificial Sequence
  The same as those in human.
ctctgagcgggaaggtgag

SEQ ID NO: 35 - DNA, 19 bp, Artificial Sequence
  The same as those in human.
aaaaaagcggagacaggag

SEQ ID NO: 36 - DNA, 19 bp, Artificial Sequence
  The same as those in human.
ccatcccgacctcgcgcta

SEQ ID NO: 37 - DNA, 19 bp, Artificial Sequence
  The same as those in human.
gtttctacgggaaatcatt

SEQ ID NO: 38 - DNA, 19 bp, Artificial Sequence
  The same as those in human.
cgccattgcacgtgccctg

SEQ ID NO: 39 - DNA, 19 bp, Artificial Sequence
  The same as those in human.
tccagtcggatgtctactc

SEQ ID NO: 40 - DNA, 19 bp, Artificial Sequence
  The same as those in human.
tcagcgccgggcatcagat

SEQ ID NO: 41 - DNA, 19 bp, Artificial Sequence
  The same as those in human.
ctttgctcggaagacgttc

SEQ ID NO: 42 - DNA, 19 bp, Artificial Sequence
  The same as those in human.
aagagagcgggcaccagta

SEQ ID NO: 43 - DNA, 20 bp, Artificial Sequence
  The same as those in human.
tcccgcctgtgacatgcatt

SEQ ID NO: 44 - DNA, 19 bp, Artificial Sequence
  The same as those in human.
cttcgagcggatccgcaag

SEQ ID NO: 45 - DNA, 19 bp, Artificial Sequence
  The same as those in human.
gaggtgtcggaccgcatca

SEQ ID NO: 46 - DNA, 19 bp, Artificial Sequence
  The same as those in human.
catgttccgggacaaaagc

SEQ ID NO: 47 - DNA, 19 bp, Artificial Sequence
  The same as those in human.
acaactacggagttgccat

SEQ ID NO: 48 - DNA, 19 bp, Artificial Sequence
  The same as those in human.
tcaaagtcggacagcctca

SEQ ID NO: 49 - DNA, 19 bp, Artificial Sequence
  The same as those in human.
gtttctgcggatgcttctg

SEQ ID NO: 50 - DNA, 19 bp, Artificial Sequence
  The same as those in human.
ctcttagcggttatccacg

SEQ ID NO: 51 - DNA, 19 bp, Artificial Sequence
  The same as those in human.
atgaccgggagtcgtggcc

(19) United States
(12) Patent Application Publication
(10) Pub. No.: US 2004/0072769 A1
(43) Pub. Date: Apr. 15, 2004

(54) Methods for design and selection of short double-stranded oligonucleotides, and compounds of gene drugs

(76) Inventor: James Qinwei Yin, Boston, MA (US)

Correspondence Address: James Qinwei Yin, First Floor, 17 Howell Street, Boston, MA, 02125, US

(21) Appl. No.: 10/016,490

(22) Filed: Sep. 16, 2002

(51) Int. Cl. C12Q 1/68; G06F 19/00; G01N 33/48; G01N 33/50; A61K 48/00

(52) U.S. Cl. 514/44; 435/6; 702/20

(57) ABSTRACT

The present invention provides methods for designing and selecting efficacious SDSOs as a gene drug that can specifically inactivate a group of corresponding genes. In particular, this invention relates to a process including the recruitment of target genes causing a disease, the identification of an endogenous siRNA sequence, the prediction of an efficacious SDSO, and the assembly of one or more SDSOs into related carriers with the ability targeting to diseased a cell or a tissue. This invention further includes pharmaceutical compounds of a gene drug, particularly one or more 21 nt double-stranded oligonucleotides with a 5′-AU(T)CCG-3′ or 5′-U(T)CCCG-3′ cleavage pattern in its antisense strand, which can specifically hybridize with a 5′-CGGAU(T)-3′ or 5′-CGGGA-3′ motif in a or more cognate RNA molecules such as a primary transcript or an mRNA. Methods of using these compounds for treatment of diseases or disorders associated with expression of one or a group of genes in a cell or tissue of the human or other animals are also provided.

### FIELD OF THE INVENTION

The field of the invention is short double-stranded oligonucleotides, and a process for manufacturing gene drugs.

### BACKGROUND OF THE INVENTION

New Technologies

The advent of the computer chip makes us embed our talents in everything from missiles, to the internet, to palm computer while biochips using photolithography, the same technique that makes the world's microprocessors, are bring us into the genomic world from the gene sequence of living thing, to the cause of cancer, to the prevent of aging (Pandey, A. et al. 2001, Nature 405:837-846; Shoemaker, D D et al., 2001, Nature 409:922-927). With the combination of computer science and biology, scientists have finished the Human Genome Project, unraveling the alignment of the 3.2 gigabase of human genome, identifying a large number of repeat sequence, and calculating about 32,000 genes embedded in less than 5% of all the human DNA sequences. Based on this great achievement, the human genome SNP map has been made with 1.42 million single nucleotide polymorphisms (SNP) identified and localized (The international SNP map working group, 2001, Nature 409:928-933). In the daily scientific activity, bioinformatics approaches such as Blast and Fasta can facilitate scientist to align sequences, compare homology, identify sequence patterns, and find out motifs (Brown S A, 2000, Bioinformatics Eaton Publishing). Marrying these biometric hands to the fast increasing body of information from functional and structural genomics is paving a wide and bright highway for designing a broad spectrum of gene drugs to the functional targets of genomics.

These world-changing chips give medical researchers the ability to analyze thousands of genes at once—in effect, to speed-read the book of life. The merging of gene sequencing and gene chip technologies makes scientists to understand that a group of aberrant genes make cancer cells different from normal cells. Recent headlines on single genes that cause rare inherited diseases will pale beside tomorrow's on patterns of genes predisposing us to heart attacks or Alzheimer's disease (Marcotte, et al, 2001, Trends in Pharmacological Science 22:426-437). Most dramatic will be the impact on the $200-billion-a-year worldwide pharmaceuticals business. New generations of drugs will increasingly be tailored to particular patients and will aim not only at treating disease but also at preventing it (Lockhart, et al., 2000, Nature 405:827-838). More importantly, it will bring out a pharmaceutical revolution, making big changes in drug forms, targets and compositions.

If gene chip microarrays allow one to simultaneously identify the genes that are expressed in a given tissue that enables one to discern the full spectrum of events operating in the disease process, bioinformatics empower one to find out specific motif and sequence patterns that include crucial cleavage sits as the reliable indication for drug target and drug itself. With the human genome fully mapped, the gene database could be an important tool for searching genomic information, comparing conservation domains between different species and identifying disease genes by way of linking and mining their data and DNA profiles. More and more websites begin to establish particular databanks on genes involved in common diseases such as cancer, diabetes, neurology, AIDS, and heart disease (Marcotte, et al, 2001, Trends in Pharmacological Science 22:426-437). The key benefits that genomics brings to us is the direct identification of therapeutic targets from the genome sequence, rather than from proteins characterized and crystallized on the basis of their biological functions. Obviously, the next generation of biotech medicine may be the fruit of mining the human genome for functional proteins, rather than only a way to targeting protein activities.

The question of why cancers are so hard to be cured by using current drugs and/or therapeutic options, but an answer may not be far from us. New gene chip technology using a DNA microarray will allow medical researchers to analyze the expression of up to 65,000 genes from cancers. The data will be compared to the normal cells, and can be quickly analyzed by computer. Furthermore, the interaction of drugs and their targets can be simulated through computational method. Excitingly, many promising gene therapies are being designed and developed. Scientists have become to realize that a 19-25 nt oligonucleotide can really inactivate its cognate RNA (Lockhart, et al., 2000, Nature 405:827-838). A central attention has been paid to how to identify and localize the target fragment of a mRNA sequence.

Now it has become clear that the natural function of RNA interference (RNAi) process is ancient protective system of biological genome against invasion by mobile genetic elements such as transposons and viruses. RNAi, the oldest and most ubiquitous antiviral system, is closely linked to the post-transcriptional gene-silencing mechanism in plants and quelling in fungi and animals. RNAi was also observed subsequently in insects, frogs, mice, rats, chicken, and human beings. In the recent experiments, a gene for luciferase, the enzyme that gives fireflies their eerie glow was introduced into a range of mammal cells, including human embryonic kidney tissue, Hela cells and Chinese hamster tissue. 19-25 nt small interference RNAs (siRNAs) introduced into these cells were able to efficiently reduce the functioning of the luciferase gene (Carthew, R. W. (2001) Curr. Opin. Cell Biol. 13, 244-248; Bernstein, E., et al., (2001) Nature, (London) 409, 363-366; Tuschl, T., et al., (1999) Genes Dev. 13, 3191-3197. Oelgeschlager, M., et al., (2000), Nature, (London) 405, 757-763). Subsequently, RNAi were proved to be also effective at targeting several naturally occurring genes such as pkc-alpha, ras, cdk-2, mdm-2 bcl-2, or/and vegf in the cells from the patient with melanoma or squamous cell carcinoma (unpublished data).

New Markets

The discovery of novel bio-drugs by the pharmaceutical industry has been motivated by several factors.

First, an increasing number of virus and fungal infections have been observed worldwide in the past decade,

Second, the number of anticancer drugs available to treat cancers in humans remains limited to a few agents, but effectiveness is not obvious,

Third, increasingly encountering natural or acquired resistance to chemical drugs and their toxic side effects are often reported,

Forth, no specific and effective drugs are available in controlling genetic diseases.

The abnormal expression of genes in human body is the main cause of many diseases from exogenous viral, bacterial, and fungal infection to endogenous hyperlipoproteinemias, cancer, hypertension, Alzheimer's, and other inherited diseases. The most important goal of medicine and healthcare is to find ways of stopping it from working in order to control the development and spread of diseases effectively, and to cure them completely and thoroughly. Naturally, a large number of diverse and talented scientists and pharmaceutical companies are working on these problems, and exploring other promising form of therapy. Gene drugs are doubtless becoming next generations of big apple in pharmaceutical industry.

It is now clear that novel genetic technologies are needed to provide greater insight into the molecular mechanisms of diseases. Scientists have used a combination of RNA inhibition and promoter interference to identify genes critical for the growth of viruses, fungi, and bacteria, the cancer genesis, and the origin of genetic disease. Naturally, when these genes are used as targets, their cognate RNA molecules will be the most effective drugs. Drug discovery based on this approach will have the huge potential to facilitate the identification of specific targets with unique modes of action, and lower the cost of research and development of corresponding drugs.

An understanding of the structural interaction between a drug and its target molecule often provides critical insight into the drug's mechanism of action. The most reliable way to assess this interaction is to use experimental methods to solve the structure of a drug-target complex. Once again, these experimental approaches are expensive, so computational methods are playing an important role. Typically, we can assess the physical and chemical features of the drug molecule and can use them to find complementary regions of the target. For example, a highly electronegative drug molecule will be most likely to bind in a pocket of the target that has electropositive features. Obviously, gene drugs can perfectly solve all the difficulty problems puzzling drug designers and shorten the R&D period.

If the interest in RNA as a drug target is owing to some of the advantages RNA over more traditional protein targets, the strategic development of RNA as a drug might be that RNA is much superior to many other bio-drugs. In addition, the raw DNA sequence information gained from the Human Genome Project brought with it a wealth of RNA data we did not have before. Researchers could not have tackled searching all the genomes of all organisms in pursuit of sequence structures and comparing a huge amount of fragments of DNA genomic sequences without today's sophisticated computational tools. When all this essential conditions and factors come together, it is the time when a new type of gene drugs appears on the horizon of pharmaceutical industries.

RNA is a rather unique class of targets because it is the only biomolecule with the dual property of carrying genetic information (similar to DNA) and of displaying catalytic activities (like protein enzymes). Similar to proteins, RNA achieves its biological function by adopting specific 3-D structures, often stabilized by proteins or small co-factors. The different forms of oligonucleotides have the potential to function as highly selective therapeutic agents by virtue of their ability to bind with unique nucleotide sequences in mRNAs for disease-causing proteins, including those implicated in cancer, virus infection and genetic disease and for other biological ends.

Three basic strategies have been developed for designing gene therapy, in which three different RNases were employed. They are RNase-L, RNase-H and RNase-III. These enzymes can break down corresponding RNA molecules aimed by a special oligonucleotide, resulting in the functional failure of those RNAs. Because activation of different nucleases needs different types of oligonucleotide as their activator, it has been revealed that 2-5A molecule, cDNA and dsRNA can activate RNase-L, RNase-H and RNase-III, respectively. Generally speaking, RNase-L can inactivate single-stranded mRNA, RNase-H can break down double-stranded mRNA (cDNA-mRNA), and RNase-III can silence triple-stranded mRNA (dsRNA-mRNA). Targeting mRNA is attractive because mRNA is more accessible than the corresponding gene. The most familiar way is to introduce antisense nucleic acids into a cell where they will form Watson-Crick base pairs with the targeted mRNA. Hybridized mRNA cannot play its function, and finally RNase H, a cellular endonuclease, which cleaves the RNA strand of an RNA-DNA duplex, will degrade the duplexed mRRA. Activation of RNase H, therefore, results in cleavage of the RNA target, thereby enforcing the efficacy of inhibiting gene expression by antisense DNA. Although a number of research work and clinical trial have been carried out, it is perhaps not surprising that effective and efficient clinical application of the antisense strategy has proven elusive. While a number of phase I/II trials employing antisense RNA have been reported, virtually all have been characterized by a lack of toxicity but only modest clinical effects. The main question is that those antisense RNAs introduced into cells typically tail off their activity after only a short time.

The second strategy is to make a 2-5A-antisense chimera, which has the general formula sp5′A2′[p5′A2′]3O(CH2)4OpO(CH2)4Op5′(dN)m, and are abbreviated 2-5A4-Bu2-(dN)m. The 5′ terminus of the 2-5A moiety bears a 5-monothiophosphoryl group, and the antisense domain is of varying nucleotide composition. 2-5A functions as a potent inhibitor of translation through the activation of a constitutive latent endonuclease, the 2-5A-dependent RNase (RNase L), which can nonspecifically degrade RNAs. Thus, when antisense RNA is coupled with 2-5A, the resulting chimerical antisense molecule empowers the cleavage specificity to RNase L. (Maitra R K,: et al., 1995, J Biol Chem 270:15071; Cirino N M, et al., 1997, Proc Natl Acad Sci USA 94:1937; Szczylik C, et al., 1991, Science 253:562; Lesiak K, et al.,. 1993, Bioconjugate Chem 4:467). Recently, scientists reported that novel chimerical antisense molecules, 2-5A-antisense can effectively control of RSV infections. The results demonstrated that 2-5A-antisense chimera has 50-90 times the anti-RSV potency of the presently employed anti-RSV therapeutic, ribavirin that is the only anti-RSV chemotherapeutic agent. However, its stability and specificity remained to be proven and improved.

The third newly developing approach that the invention prefers to emphasize is a RNA interference (RNAi) technology. RNAi has been found in many organisms including plants, protozoa, nematodes, insects, animals and human. RNAi is the oldest and most ubiquitous protective system in the cellular level. Through thousands and thousands of evolution and natural selection, this system still exists in cells of different species, suggesting its importance in biological function. RNAi employs a gene-specific double-stranded RNA. The dsRNA can be transferred into a serial of short interfering RNA (siRNA) under the action of RNase III. A siRNA bound to RNase III can bring the latter to a region of an mRNA that is complementary to the antisense strand of this siRNA. Subsequently, RNase III is able to break specifically down the mRNA molecule (Fire, A. & Mello, C. C. (1999) Cell 99, 123-132; Cogoni, C. & Macino, G. (2000) Curr. Opin. Genet. Dev. 10, 638-643; Matzke, M. A., et al., (2001) Curr. Opin., Genet. Dev. 11, 221-227; Zamore, P. D., Tuschl, T., Sharp, P. A. & Bartel, D. P. (2000) Cell 101, 25-33).

By borrowing the seed selected by nature, the invention attempt to enhance and enlarge this ancient protective system in vitro, and then introduce therapeutic amount of siRNA molecules into those abnormal cells in order to silence corresponding mRNAs. Thus, the active agents of gene drugs of the invention, a type of natural siRNA molecules, possess many advantages over other gene therapy or drug treatment. These merits include but are not limited to:

Brand-new therapeutic mechanisms: siRNAs naturally-occurring in the living things are employed as gene drugs for the treatment of diseases,

High resistance to nuclease: 19-25 nt double-stranded oligonucleotides are stronger resistance to nucleases than single-stranded oligonucleotide,

Long-term biological effects: siRNA may be amplified and spread through possible replication mediated by RNA polymerase, and the possible methylation of cognate DNA sequence may cause the suppression of corresponding gene,

High specificity: the siRNA obtained by the computational selection is not significantly homologous to any other genomic DNA sequences,

High cutting efficacy: all the siRNA employed by the invention have at least two strong cleavage sites of RNase III,

High effectiveness: one or more kinds and classes of different 19-25 nt double-stranded oligonucleotides may mix together, and each one has its unique biological function and action mode for the degradation of many target oligonucleotides at the same time,

High resistance to mutant: mutant probability occurring in a 19-25 nt sequence is much less than that in a longer sequence from several hundreds to thousands of bases.

Based on the prior successes and failures in gene drug discovery and clinical application, the invention focuses on employing many advanced technologies, and developing new and comprehensive compounds and compositions of gene drugs.

### BRIEF SUMMARY OF THE INVENTION

The present invention integrates computer technology, RNA interfering technology, gene engineering, gene-chip microarrays, and human genome databases into the process for manufacturing of gene drugs. The two main objects of the present invention are described as follows:

to provide a general process for the recruitment, selection, syntheses, purification, compound, and assembly of a new type of gene drugs used for the treatment of different viral infections, cancers and genetic diseases of a human or an animal, in which a simplified method for predicting an efficacious SDSOs is particularly emphasized.

and to describe compounds of different gene drugs, particularly 21-25 nt double-stranded oligonucleotides with a particular cleavage pattern CGGAU, CGGGA or their derivatives, which are targeted to their homologous nucleic acids, and employed to modulate expression of corresponding RNA molecules and possible methylation of cognate DNA sequences.

Pharmaceutical and other compositions comprising the compounds or compositions of the invention are also described in details. Further provided are methods of treating an animal and a plant, particularly a human, predisposed to a disease or condition associated with expression of one or more given protein by administering a therapeutically or prophylactically effective amount of one or more 20-25 nt double-stranded oligonucleotides of the compounds or compositions of the invention

A group of 20-25 nt double-stranded oligonucleotides with a specific cleavage pattern designed and developed as main active agents of gene drugs of the invention include the following advantages:

1. brand-new design and production principles—a naturally-occurring RNA interfering protection system within a cell is specifically amplified and enhanced with bioengineering technology, and then it can be used to inactivate homologous target RNA molecules, particularly mRNAs. The pattern CGGAU, CGGGA or their derivatives, a cluster of strong cleavage sites, is used as the basis for selecting and designing gene drugs;

2. short period of drug discovery—with the assistance of computer and gene-chips, selecting the most potent motif within a given mRNA sequence as a drug target and its cognate partial sequence as a drug can greatly decrease the time used to study chemical features of the drug molecule and to find its complementary regions of the target;

3. low cost of drug discovery—because a study of the structural interaction between a drug and its target molecule often needs higher experimental expenditure and longer time, fast computational method and established gene databases used in gene drug design of the invention will remarkably reduce the R&D cost;

4. high specificity—the most potent target portion within a given mRNA sequence can be predicted and selected, and the typical Watson-Crick base-pair principle is embedded in the therapeutic mechanisms of gene drugs of the invention;

5. less toxic and side effects—because critical compositions of gene drugs of the invention exist naturally in the organisms and their high specificity and effectiveness bring the need of low dose, their toxic and side effects can be much lower than other chemical drugs designed by a man;

6. good stability—double-stranded oligonucleotides have much better stability because they have stronger ability against related nucleases, good capacity to bind to related proteins or small co-factors, and some bases easy to be modified;

7. flexible usage—the combination of different types and amounts of double-stranded oligonucleotides can make diverse therapeutic effects according to the requirements and needs of patient or disease status;

8. high effectiveness—inactivating more than one specific mRNAs at the same time is the most important merit of the gene drugs of the present invention, compared to other single gene therapy and chemical drugs. The methodological breakthrough particularly benefits for cancer therapy.

9. high resistance to mutation owing to much less mutant probability occurring in a 20-25 nt sequence compared to a longer sequence from several hundreds to thousands of bases.

### DETAILED DESCRIPTION OF THE INVENTION

The gene drugs may soon become the leading disease-treated agents in the world. In the United States, gene therapy has been going through the research, development, clinical trials and practical application as therapeutic options, even though there are some obvious weakness such as obvious instability, and less efficacy. Many skilled workers in the art have been trying to find out appropriate approaches of making a gene drug with special efficacy and reliable stability. In order to meet the two main goals, there occurs a brand-new idea forthcoming with respect to a new type of gene drugs that is displaying our better understanding of gene therapy at the molecular level, greater focus on mRNA-based target identification, and broader use of natural and computational selection to more comprehensively evaluate potential gene drugs. With the knowledge of the human genome and the genetic basis of disease, as well as the integration of computer science, biochips, short interfering RNA (siRNA) and genomic technologies, new therapeutic approaches are being developed for the treatment of many puzzled diseases such as viral infections, cancers and genetic diseases. The approaches and compositions of the invention can be effective and safe, and ultimately provide cures. The present intervention addresses the critical elements of gene drugs and related scientific approaches, and describes the detailed process of producing gene drugs for those diseases that cannot effectively be treated by current drugs and other therapeutic options.

In the context of this invention, the term “gene drug” refers to one or more types of small double-stranded oligonucleotides (SDSO) with one cleavage pattern CGGAU embedded in a pharmaceutically acceptable carrier, whereby the SDSO can be transferred to a cell of an animal, preferably a human. The term “gene drug” further includes naked SDSOs and other agents.

As used herein, the term “oligonucleotides” means a nucleic acid-containing polymer or oligomer duplex, such as a siRNA, a sRNA-cDNA or a double-stranded DNA (dsDNA). This term further includes oligonucleotides composed of naturally-occurring nucleobases, sugars and covalent internucleoside linkages as well as oligonucleotides comprising modified or non-naturally-occurring portions. Each of these types of polymers, as well as numerous variants, is known in the art. Such modified or substituted oligonucleotides are often superior to native forms because of some desirable properties including stronger cellular uptake, higher affinity for nucleic acid target, and better resistance to nucleases.

As used herein, the term “siRNA, sRNA-cDNA or dsDNA” means a nucleic acid duplex, each strand of which is composed of 21 to 25 nucleosides. The SDSOs of the invention can inactivate their cognate nucleic acids in a normal cell or in a diseased cell. The SDSO of the invention include, but are not limited to, phosphorothioate oligonucleotides and other modifications of oligonucleotides.

As used herein, the terms “specific SDSO” means a 19-25 nt double-stranded oligonucleotides, whose sense strand is completely homologous to a specific region of all the members or at least one member of its family genomic DNA, and has less than 80% similarity of any members of other family genomic DNA. Its antisense strand can hybridize with a corresponding mRNA, and guide a RNase III to break specifically down the mRNA molecule, but other mRNA molecules. Several lines of experiments demonstrated that the difference of only one nucleoside between siRNA molecule and its cognate sequence of the target mRNA can cause the failure of that siRNA to inhibit the activity of the mRNA.

As used herein, the terms “efficacious SDSOs” mean short double-stranded oligonucleotides, which contain a cleavage center. The cleavage center is a specific sequence with the length of five nucleosides. The sequence of SDSO sense strand includes but is not limited to CGGAA, CGGAC, CGGAG, CGGAU(T), CGGGA, CGGGC, CGGGG, CGGGU(T), and other derivative sequences, while The sequence of SDSO antisense strand includes but is not limited to the sequences complementary to those in its sense strand, that is UUCCG, GUCCG, CUCCG, AUCCG, UCCCG, GCCCG, CCCCG, ACCCG and other derivative sequences. These sequences have two to three strong cleavage sites of RNase III. These sites include G*G, G*A and A*U. Thus, a SDSO molecule with two or three strong cleavage sites can break down its target mRNA efficiently and specifically.

As used herein, the terms “cognate nucleic acids” include DNA encoding protein and other functional RNAs, RNA (including pre-mRNA, mRNA, and other RNA molecules) made from such DNA, and homologous fragments of such DNA. The specific interaction of a siRNA compound with its target nucleic acid influences the normal function of the nucleic acid. This suppression of function of a target nucleic acid by its specific interaction with siRNA, or/and sRNA-cDNA and dsDNA is generally defined as “RNA or DNA interference”. The functions of RNA to be interfered with include all critical functions such as transcription of mRNA, translocation of the RNA to the site of protein translation, splicing of the RNA to yield one or more mRNA species, translation of protein from the RNA, and other special functions mediated by the RNA. The functions of DNA to be interfered with include replication, repair, recombination, and transcription. The resulting ends of such interference with target nucleic acid function are suppression of the expression of corresponding proteins, and of specific functions of other RNA molecules as well as methylation of cognate DNA sequences.

Although the two strategic goals may be met by offering SDSO compounds that specifically interact with one or more cognate nucleic acids, the invention mainly focuses on regulating the functions of genomic RNA molecules, by which related cancers, viral infections or genetic diseases can be treated and cured at the end. Preferred nucleic acid molecules of the invention include, but are not limited to, those mRNAs encoding oncogene products, growth factors (EGF, HGF, NGF, IGF-I, IGF-II, PDGF, TNF, VEGF, alpha.-FGF, beta.-FGF, TGF-.alpha, and TGF-.beta), growth factor receptors (EGF-R, FGF-R, PDGF-R, erbB2-R and VEGF-R), Bcr-Abl, intrgrins, E-cadherin, inflammatory molecules, cytokines, interleukins, interferons, telomerase, CD40L/CD40, ICAM-1/LFA-1, hyalurin/CD44, signal transfection molecules (PKC-alpha, Stat 3 and 5, CDK-2 and 4, Ras, Raf, FAK, Src, and MEK), transcriptional activators, steroid hormone receptors (i.e. estrogen (SERMs), progesterone, testosterone, aldosterone, and corticosterone), apoptosis (e.g. Bcl-2 and caspases), LDL receptor, amyloid protein, WNKs, or the like.

Identification of Target mRNA Molecules in Diseased Tissues or Cells

The availability of sequences of normal and abnormal human genes and the development of powerful biochip technology will allow for the rapid identification of these genes and their diverse expression in any diseases, and the tactical design of relevant genetic therapies. It also benefits for better understanding the all perspectives of RNAs and proteins. The active agents of compounds of the invention can be identified and selected with biochips and other approaches as well as the literature.

Biochip technology is already providing insights into cancer that would be difficult, if not impossible, to obtain by using the gene-by-gene approach. In the past years, scientist have identified changes of many gene expression patterns in a variety of cancers, including leukemia and lymphomas, prostate and breast cancers, squamous cell cancer, melanoma, brain cancer and so forth. Some skilled worker in the art can determine which cancers are likely to respond to current therapies and which aren't. In addition, the investigations are offering researchers a clue on which a group of genes, but not a single gene, are important for the development, maintenance, and spread of the various cancers, and are thus possible drug targets. Obviously, how to select the most potent target sequences within a given mRNA sequence, and assembly this group of target sequences into a gene drug is very important issues of the present invention.

Now it is becoming clear that it's possible to detect wholesale changes in gene expression patterns with powerful gene chip microarrays. More and more biochip companies are developing new generations of gene chips for identifying genes whose activity is turned up or down, and finding out which of those changes are important for cancer development and progression, searching which gene is related to genetic and metabolic diseases, and diagnosing general diseases routinely. For example, human liquid and blood can be used to specific biochips after appropriate processes so that testing a drop of saliva from a patient can tell whether the person fell ill with viral or bacterial infection, or hay fever. Similarly, a person with the family history of cancer is able to know if he/she is suffering from the cancer only through the test of his/her blood in biochips. In the clinical practice, microarrays have bee employed to compare the gene expression patterns of highly metastatic melanoma cells with those of the much less metastatic cells from which they were derived. The comparison can also identify a suite of genes whose activity was apparently turned up as melanoma cells progressed to malignancy.

The major objective of employing biochip technology in the invention is to identify which genes are up-regulated in the diseased cells and tissues, and figure out which of them are critical factors leading to a disease. Because not all the genes that express highly will produce big amount of corresponding proteins, the change in synthesis and amount of a protein may be a more important and direct index, indicating specific risk assessment with its related gene. Naturally, the combination of gene chip and protein chip in the invention will provide the testing results with their own information and synergetic effects. Taken together, comparison of the difference in the expression of genes between the normal and abnormal cells and tissues and between different diseased cells and tissues at the different stages of the disease as well as the difference in testing results between the gene and protein chips can provide invaluable information for selecting target RNA and its cognate double-stranded oligonucleotides with the 20-25 nt length as a gene drug.

Identification of Endogenous siRNAs

After obtaining related information about the target genes and their RNAs, the invention introduces a method for selecting a double-stranded oligonucleotides that is efficacious for inhibiting expression of a cognate RNA. The identification of endogenous RNA interfering gene is a critical step for selecting a specific sequence homologous to its mRNA molecules as an active agent of gene drugs, because evolutionary characteristics of an endogenous RNA interfering gene will bring us with excellent natural selection of target sequences, offer much effective and efficient cognate genomic segment, and thus save our searching time.

Although the complete human genome sequence provides a rapid inventory of most encoded proteins, tRNAs and rRNAs, it has not led to the immediate recognition of other genes that are not translated. In particular, a new type of endogenous RNA interfering genes have been overlooked because there are no identifiable classes of RNAs that can be found based solely on sequence determinants. The RNA motif, particularly stem-loop RNA motif discovery, is very useful and important because it can also be employed to detect endogenous RNAs. Except for the combined use of ready approaches such as FOLDALIGN (http://www.bioinf.au.dk/slash/) for RNA structure prediction, a set of specific software has also been developed to look for endogenous RNAi molecules, including computer searching of complete genomes based on parameters common to RNAi molecules, probing of genomic microarrays, and isolating dsRNAs based on an association with general RNA-binding proteins such as adenosine deaminases, a dsRNA binding proteins (dsRBPs). So, the first step we should take is to identify if there exist any endogenous RNA molecules in human genome, which meet the requirement of being a drug target and drug itself perfectly.

RNAi is defined as a class of RNA molecules that do not function by encoding a complete open reading frame (ORF). These RNAi genes are found to have very high conservation of sequences between different organisms. In most cases, the conservation between human and Caenorhabditis elegans was >95% (FIG. 1), whereas that of the typical gene encoding an ORF was frequently <70%. Conservation tests on random noncoding regions of the parameter to screen for new RNAi genes. It is possible for this method to be used to search endogenous RNAI in the human genome. Therefore, the invention proposes the indicative selecting an endogenous RNAi gene, including the sequence that can encode a stem-loop RNA, whose stem is high conserved, and 19-25 nt nucleosides in length, and which is localized in intron region or intergentic region.

All possible RNAi molecules may be encoded within intergenetic regions (between two genes encoding proteins) or introns regions. A difficulty is that the databases containing all intergenic sequences from genomes of different species have been not available to be used as a starting point for specific homology search. Much searching work can be carried out in the current gene databases and privileged computer software. The principle used in the software is well known in the art. A first region of a nucleic acid is complementary to a second region of the same nucleic acid if, when the two regions are arranged in an antiparallel fashion, at least one nucleotide residue of the first region is capable of base pairing with a nucleotide residue of the second region. Preferably, when the first and second regions are arranged in an antiparallel fashion, at least about 95% of the nucleotide residues of the first region are capable of base pairing with nucleotide residues in the second region. The region usually covers a 19-25 nt-nucleotide length. Most preferably, all nucleotide residues of the first region are capable of base pairing with nucleotide residues in the second region (i.e. the first region is “completely complementary” to the second region). It is known that an adenine residue of a first nucleic acid strand is capable of forming specific hydrogen bonds with a residue of a second nucleic acid strand that is antiparallel to the first strand if the residue is thymine or uracil. Similarly, it is known that a cytosine residue of a first nucleic acid strand is capable of base pairing with a residue of a second nucleic acid strand that is antiparallel to the first strand if the residue is guanine.

For example, let-7, an intergenic region was rated based on the degree of conservation and length of the conserved region when compared to the human, Drosophilae melanogaster and Caenorhabditis elegans (FIG. 6). The highest rating was given to intergenic regions with a high degree of conservation (raw BLAST score of 42) over at least 21 nt. Note that most promoters do not meet these length and conservation requirements. FIG. 1 shows a set of BLAST searches for let7 RNAi and three regions with high conservation (#1, #2, and #3). Taken together, the high conserved sequence for possible stem-loops, in particular those with characteristics of 21 nucleotide length can be considered as especially an indicative of possible RNAi genes.

In order to avoid the obstacle of nucleic membrane to siRNAs and uncertain interaction of siRNAs and other parts of a encoding gene such as introns, the borderings of ORFs the intergenetic regions and other nonencoding regions of pre-mRNA, the siRNAs which have the same sequence as the portion within a corresponding ROF are employed in a composition and compound of a gene drug of the invention.

Searching Conserved Sequence by Structural Homology Analysis

If a related endogenous RNAi molecule can not be found in the current available databases, the analysis of a family of homologous sequences has to be conducted through searching for all available members of that family. In this step, a key task is to recruit structural homologous sequences shared by most members of a gene family from different species. Structure homology is used to describe features of the three-dimensional structures of a macromolecule, and to provide information about the corresponding sequence. The highly conserved sequences (motifs) naturally selected out contain the most important genetic information, which can be constantly kept in many different species. The motifs are often composed of a combination of sequence and structural constraints such that the overall structure is preserved even though much of the primary sequence is variable. An important issue of searching specific gene segment is to find out highly conserved sequence among different species and identify specific structural patterns among different mutations of the same gene family in the different species, with maximal, if not all, non-similarity to any other genes. In the case of inactivation of all the member mRNAs of a oncogene family, it is necessary to identify specific sequence patterns shared by all the members of the same family. Thus, when selected sequence is designed as a gene drug, it can initiate a specific degradation process against all the cognate genomic RNA molecules of that gene family. This method also benefits for treating different patients with the same disease-causing gene but different SNP status. FIG. 2 and FIG. 3 show a typical example.

Multiple alignment programs can detect motif patterns on the same gene family in several different species. For more than two sequences, heuristic approaches have generally to be employed. Usually, the multiple alignment should be carried out first with a progressive alignment program. These programs are fast, do not need large memory capacity and may thus be run on large dataset even on microcomputers. Among programs using this approach, MUSCA (http://cbcsrv.watson.ibm.com/tmsa.html) and CLUSTAL W (http://www2.ebi.ac.uk/clustalw/) are the best to be used to finish this tough work. CLUSTALW can also run on a specified region and/or a specified set of sequences, without changing the rest of the alignment. If this first alignment shows that all sequences are related to each other over their entire lengths. It is unlikely that any other method will give a better result. The sequences used in the invention were compiled from various sources databases using the Blast algorithm. A multiple sequence alignment of most members of a IGF-2 gene family from different species was made using CLUSTAL W. The resulting multiple sequence alignment was manually refined to display the common high conserved region. A final data set of human IGF-2 was selected for the further analysis (FIG. 3 and FIG. 4).

However, if there are some highly divergent sequences, large gaps, or poorly conserved regions, it is recommended to compare the results of different methods and/or sets of parameters. FIG. 5 shows homologous sequences sharing conserved blocks separated by non-conserved regions of varying size. This situation, which is frequently observed in genomic DNA sequences, is particularly error prone for progressive alignment methods, notably because the linear weighting of gaps tends to over-penalize long indexes. The two-sequence alignment of BLAST is the best way to solve this kind of problem. Weighting sites according to their degree of conservation may improve the sensitivity of a sequence similarity search. Thus, once several homologous sequences have been identified, it is possible to use methods such as profile searches BLAST that rely on a multiple alignment to identify more distantly related members of the family (Brown et al, 2000, Bioinformatics Eaton Publishing; Higgns et al, 2000 Bioinformatics. Oxford University Press; Durbin et al, 1998, Biological sequence analysis. Cambridge University Press).

Selecting Candidate Sequence by Human Sequence Pattern Analysis

In this section, it is necessary to figure out which highly conserved sequences are shared not only by this family also by other families in human being. A way to analyze the sequences is to group them into families, each family being a set of sequences, which are evolutionarily, structurally, or functionally related, and conserve their common features or patterns. It is suggested that highly conserved DNA sequences are invariably involved in an important function, while sequence patterns can be used to discriminate between family members and nonmembers. A combination of pattern discovery algorithms with rigorous multiple alignment between many member sequences of a gene family may provide an effective method for identifying critical segment in both this family and other families, or only in this family but not in other families. Finally, this constant pattern only contained in a single family, not shared by other families will be used as a potentially active agent of gene drugs of the invention.

To detect DNA sequence homology, BLAST and FASTA searches can be used against the SWISS-PROT, EMBL and GenBank databases where published nucleic acid sequences are stored, organized, and managed. However, it is not possible to rely on the annotation to identify in a database all homologous sequences belonging to a given family. Presently, the most efficient way to identify those homologs consists in taking one member of the family and comparing it to the entire database with a similarity search program such as FASTA, BLAST or BUST. In an independent series of experiments, a specific DNA sequence such as IGF-2 was used to detect transcripts that might correspond to the siRNA from a RNA region which encoding an IGF-2 protein. The indicated sequences are used in a BLAST search of the NCBI Homo Sapiens Genomes database. To guarantee a more exhaustive search, one may repeat this procedure with several distantly related homologs of different species identified in the first step. After running the query, the Blast will indicate how many sequences have been scanned over, and how many hits have been found. In the results of Blast, sequences producing significant alignments are listed in the order of score. According to the differences in the score, different groups of sequences with most similarity can be sorted out. The number of members in the same family and other families can be counted. Comparison of different queries, the best sequence will be selected with minimal similarity to other sequences, and the number of all the listed sequences is also minimal among all the queries (FIG. 4A and FIG. 4B).

Selecting SDSO Sequence by Specific Cleavage Pattern

Another question about a specific sequence of the invention is the number and order of nucleotides in the sequence and specific pattern. Purine-rich oligonucleotides, especially ones containing four consecutive guanine residues, have a tendency to form stable tetrameric structures under physiologic conditions. The guanines of single-stranded oligonucleotides are not restrained in space by rigid double-helix structure and can therefore form various hydrogen bonds not observed in Watson-Crick base pairing. Tetraplexes known as G quartets arise as a result. Dissociation rates of these structures may be quite slow and may prevent hybridization of the oligonucleotides to their target transcript, rendering them ineffective as the active agents of gene drugs. Another interesting issue of nucleotides is that RNase III seams to have a favor with uracils. So, more U bases in 19-25 nt oligonucleotides seems to enhance the binding ability to a RNase.

The specific binding and high cleavage rates are the most important issues for designing and selecting an efficacious SDSO. The invention combines a cluster of strong cleavage sites and the specific sequence shared by most members of the same gene family and lest members of other families, and provides a simplified method for accurate prediction of a highly efficient SDSO, which contains a cleavage center. The cleavage center includes a set of cleavage patterns comprising CGGAU(T), CGGGA and their derivatives. Several lines of studies demonstrated that RNase III preferred to make a strong cleavage at GG, GA, or AU position, while CGG may be a favorable position for the methylation of DNA sequence. The cleavage pattern of the invention will benefits not only for saving time in searching specific sequence (FIG. 7), but also for paving a path to investigate the regulation of genomic functions.

The careful analysis of a cleavage pattern demonstrated that each pattern bears three strong cleavage sites such as GG, GA, and/or AU, and contains a critical core, that is CGG. The CGG is very conserved and important compositions. If it is changed, the specificity of a SDSO will be altered. Generally speaking, the nonspecific matches or partially complementary sequences will rise in most cases. The derivatives of a cleavage pattern mainly come from the changes occurring in the fourth and fifth letters. Even though the fourth position can be taken by A, C, G, or U, preferred letters are A and G in most cases. Several lines of experiments demonstrates that A and G are capacity of forming the second strong cleavage site with a G the third position, and the selected sequence has higher specificity. Similarly, the fifth position also has a favor of a letter, that is U (T) and A, constituting the third strong cleavage. All the useful cleavage patterns include but are not limited to CGGAU (T), CGGAA, CGGAC, CGGAG, CGGGA, CGGGC, and CGGGU (T). Taken together, the merging the CGG pattern and the characterized cleavage sites provides a very good indication for designing an efficacious SDSO (FIG. 7).

The particular cleavage pattern of oligonucleotides of the invention is CG*G*A*U (T) in the most sense strands, and GCCU (T) A in the most antisense strands (where G*G, G*A and A*U are strong cleavage sites). The position of the second G and corresponding C should be located near center of short strand, about 10 or 11 nt downstream of the first nucleotide that is complementary to the 21 nt to 23 nt guide sequence. The core of pattern is CGG that is closely related to the specificity of small double-stranded oligonucleotides, while other two nucleotides can be replaced in the substitution manner under some conditions. The other portion of sequence of a SDSO molecule may be related to the sensitivity of the SDSO (Table 1 to 4, and Table 9 to 15).

Simplified Method for Selecting an Efficacious SDSO

The invention also includes a simplified method for predicting whether a 21 nt double-stranded oligonucleotides will be efficacious for inhibiting expression of a gene. The method focuses on determining whether the antisense strand of small double-stranded oligonucleotides is complementary to a specific portion of an RNA molecule corresponding to the gene, wherein the sequence comprises a CGGAT, CGGGA pattern or their derivatives.

The first step is to recruit which sequence of a given genomic DNA includes a 5′-CGGAT-3′ sequence or other cleavage patterns (hereinafter referred to as “CGGAT pattern”) in the sense strand of 21 nt double-stranded oligonucleotides. Accordingly, the antisense sequence of a SDSO molecule has nucleotide sequences comprising at least one copy of the sequence 5′-AU(T) CCG-3′ (hereinafter referred to as a “AU (T) CCG” pattern) which is complementary to a corresponding RNA of the genomic DNA sequence. The second step is to localize the second G and its complementary C of the cleavage pattern in the tenth or 11^{th} position of a SDSO molecule. The third step is to extend 7 nucleosides to both sides from the cleavage center, or take the sequence with the length of 19 nucleosides out the genomic DNA sequence. The forth step is to align it with other genomic DNA sequence in the human database of Genebank. The fifth step is to compare all the reaching results, and select the best one which has excellent specificity and sensitivity as candidates. The final step is to chose a SDSO molecule out from candidates as active agent of gene drug according to disease's features and patient's status. If it is not very good, the second or third sequence with a cleavage pattern should be checked up until the best one is found out. In the very few cases, the complex method introduced above can be a final backup.

It has been discovered that the sequence with a cleavage pattern in its center can display high specificity with minimal similarity to other gene sequences (Table 1 to 4 and FIG. 8). It was further revealed that the presence of the cleavage pattern in an oligonucleotide duplex is a reliable indicative that the 21 nt oligonucleotide duplex has strong inhibitory efficacy on expression of its cognate RNA (FIG. 8 and Tables 9 to 15). Thus, a cleavage pattern in an RNA molecule can be highly recommended as the basis for designing an efficacious SDSO molecule. Recognition of the significance of the AU (T) CCG pattern in efficacious 21 nt double-stranded oligonucleotides represents a significant progress over the previous design methods. The presence of the CGGAU (T) pattern in a 21 nt double-stranded oligonucleotides homologous to an RNA molecule is an indication that the 21 nt double-stranded oligonucleotides will shut off the synthesis of protein encoded by the RNA molecule efficiently. By the way of examples, the invention describes the detailed application of this method in tables 1 to 4 as well as tables 9 to 15.

The following tables show the examples obtained by using a designed cleavage pattern to select a DNA sequence as a 19 nt double-stranded oligonucleotides. Oligonucleotides having the cleavage pattern indicated in tables were selected and used to fish other complete or partial similarities as described herein. The specificity of a selected SDSO was assessed following alignment of the sequence with a cleavage pattern in Blast reaches against homo sapiens database. The match extent of a given sequence reported in Table 1 can be grouped into three different cases; That is 100% match, 80-95% match and less than 80% match. Each SDSO in Table 1 is reported using a SEQ ID NO, a 100% match, a 80-95% match and a less than 80% match, cleavage pattern and a sequence listing and an indication of the region of the sequence, to which the SDSO was selected to be complementary. “M” denotes a member of the same gene family, while “n” means a non-member of this gene family. The number under each title denotes how many member sequences or non-member sequences can be fished out from about 960,000 human genomic sequences. These sequences are completely or partially homogenous to the selected sequence. According to the data obtained, skilled workers are able to estimate how well the sensitivity or specificity of designed SDSO.

In the table 1, it demonstrated that the core of cleavage center is composed of CGG motif. If the first nucleotide, C of the core is substituted by others such as A, G, or T, the total hit will be higher.

TABLE 1
gi|14780094: Homo sapiens amyloid beta (A4) precursor protein

| Seq. | Total | 100% | 80-95% | <80% | Cleav. | Start | Sequence | End |
|---|---|---|---|---|---|---|---|---|
| ID# | Hits | Match | Match | Match | Pattern | Point | (19 Bases) | Point |
| 1 | 120 | 10 m | 2 n | 108 n | aggtc | 1 | atgtcccagg tcatgagag | 19 |
| 2 | 56 | 17 m 3 n | 1 n | 35 n | cggag | 756 | atcaagacggaggagatct | 774 |
| 3 | 205 | 16 m 3 n | 8 n | 178 n | atgca | 1079 | tgagcagatgcagaactag | 1097 |
| 4 | 248 | 15 m 4 n | 8 n | 221 n | aggat | 454 | gagattcaggatgaagttg | 472 |
| 5 | 205 | 19 m 4 n | 11 n | 161 n | tggat | 789 | g tgaagatgga tgcagaat | 807 |
| 6 | 505 | 14 m 4 n | 7 m 39 n | 441 n | gggaa | 16 | agaga atgggaagag gcag | 34 |
| 7 | 18 | 13 m 4 n | | 1 n | cggaa | 542 | tcagttacg gaaacgatgc | 460 |

The table 2 showed that sequences fished out by a VEGF sequence with the CGGAT cleavage pattern is much better in specificity than those with other different cleavage patterns, and has an equal level of sensitivity to others.

TABLE 2
gi|15422108: Homo sapiens vascular endothelial growth factor (VEGF)

| Seq. | Total | 100% | 80-95% | <80% | | Start | | End |
|---|---|---|---|---|---|---|---|---|
| ID# | Hits | Match | Match | Match | Pattern | Point | Sequence | Point |
| 1 | 201 | 22 m 4 n | 5 n | 170 n | ttggg | 21 | tgctgtcttg ggtgcattg | 39 |
| 2 | 81 | 16 m | 5 n 4 m | 56 n | tgaca | 551 | gcagatgtga caagccgag | 569 |
| 3 | 59 | 18 m | 1 n | 40 n | gaggg | 261 | caatgacgag ggcctggag | 279 |
| 4 | 23 | 21 m | | 2 n | cggat | 315 | gattat gcggatcaaa cct | 333 |
| 5 | 157 | 21 m | 20 n | 116 n | tcatg | 121 | gtgaagttca tggatgtct | 139 |
| 6 | 520 | 22 m | 11 n | 487 n | gttcc | 481 | tgtaaatgtt cctgcaaaa | 499 |
| 7 | 102 | 21 m | 4 n | 77 n | gccat | 148 | agctactgccatccaatcg | 166 |

The table 3 and 4 take BCL2 and PRKWNK4 as examples for describing the importance of the cleavage center in selecting a specific sequence from BCL2 and PRKWNK4 genomic DNA. Careful observations can find out the rule that the nucleotide in the forth position of cleavage center could be any one of four natural nucleotides. However, A and G are the best option because they can form the third strong cleavage site, and have high probability in predicting a specific SDSO molecule. Although a good SDSO molecule can sometimes be selected when C or T takes the forth position of the cleavage center, there is a big probability in fishing out a nonspecific sequence such as Seq. ID 3, 4 and 5 in table 3 and Seq. ID 14 and 15 in table 4.

TABLE 3
gi|13646672: Homo sapiens B-cell CLL/lymphoma 2 (BCL2)

| Seq. | Total | 100% | 80-95% | <80% | | Start | | End |
|---|---|---|---|---|---|---|---|---|
| ID# | Hits | Match | Match | Match | Pattern | Point | Sequence | Point |
| 2 | 18 | 8 m | 3 m | 7 n | cggtc | 187 | cggg acccggtcgc cagga | 205 |
| 3 | 152 | 11 m | 5 n | 136 n | cggct | 217 | caga ccccggctgc ccccg | 235 |
| 4 | 81 | 11 m | | 70 n | cggtg | 256 | ctcag cccggtgcca cctgtg | 276 |
| 5 | 89 | 11 m | | 78 n | cggtg | 388 | ttt gccacggtgg tggagg | 406 |
| 6 | 25 | 6 m | | 19 n | cggcc | 599 | aa ctgtacggcc ccagcat | 617 |
| 7 | 41 | 10 m | | 30 n 1 m | cgggg | 372 | caccgcgcg gggacgcttt | 390 |
| 8 | 35 | 8 m | 2 n | 22 n 3 m | cgggc | 120 | cccgcaccggg catcttct | 138 |

The table 4 systematically compared the difference in predicting efficacious sequences by the different derivatives of the cleavage pattern by taking homo sapiens protein kinase as a testing case. The results demonstrated that there was the possibility for high hits if the fourth letter within the cleavage pattern was T or C. For example, sequences 14 and 15 in SeqID#4 got high hits and more homologs of other gene families. So, the preferred cleavage pattern as a reliable prediction indicative should be one of derivatives of CGGA or CGGG.

TABLE 4
gi|15277311: Homo sapiens protein kinase, lysine deficient 4(PRKWNK4)

| Seq. | Total | 100% | 80-95% | <80% | | Start | | End |
|---|---|---|---|---|---|---|---|---|
| ID#4 | Hit | Match | Match | Match | Pattern | Point | Sequence | Point |
| 1 | 13 | 4 m | 1 n | 8 n | cggaa | 1029 | gggaccccggaattcatgg | 1047 |
| 2 | 12 | 3 m | | 9 n | cggaa | 366 | aaggctgcggaagactccg | 384 |
| 3 | 21 | 3 m | 7 n | 11 n | cggaa | 632 | gcagactcggaaactgtct | 650 |
| 4 | 24 | 3 m | 3 n | 18 n | cggac | 270 | gatcctccggactccgctg | 288 |
| 5 | 66 | 3 m | 1 n | 62 n | cggac | 393 | gagctcccggactctgcag | 411 |
| 6 | 44 | 3 m | 5 n | 36 n | cggag | 30 | ccggccacggagaccaccg | 48 |
| 7 | 12 | 3 m | | 9 n | cggag | 2193 | ctgccttcggagcgagatg | 2211 |
| 8 | 5 | 4 m | | 1 n | cggat | 1254 | atccgcacggataagaacg | 1272 |
| 9 | 7 | 3 m | | 4 n | cggat | 1752 | accacttcggattgcgaga | 1770 |
| 10 | 4 | 3 m | | 1 n | cggat | 2216 | tctcagacggattcgggag | 2234 |
| 11 | 56 | 4 m | | 52 n | cggca | 653 | agctgagcggcagcgcttc | 671 |
| 12 | 6 | 4 m | | 2 n | cggca | 1093 | acgcgttcggcatgtgcat | 1111 |
| 13 | 53 | 2 m | 1 n | 50 n | cggcc | 24 | caatccccggccacggaga | 42 |
| 14 | 136 | 3 m | 5 n | 128 n | cggcc | 2990 | tcctgctcggcccctccca | 3008 |
| 15 | 128 | 3 m | 2 n | 123 n | cggcg | 458 | cctagagcggcggcgggag | 476 |
| 16 | 171 | 3 m | 1 n | 167 n | cggcg | 1397 | ggacgcgcggcgcgggggg | 1415 |
| 17 | 34 | 3 m | | 31 n | cggct | 1872 | ctgccctcggcttttgccc | 1890 |
| 18 | 66 | 3 m | 2 n | 61 n | cggga | 151 | gcttctccgggaaggctga | 169 |
| 19 | 48 | 4 m | 3 n | 41 n | cggga | 911 | cctgcaccgggatctcaag | 929 |
| 20 | 15 | 4 m | | 11 n | cggga | 942 | tttatcacgggacctactg | 960 |
| 21 | 72 | 3 m | 1 | 68 n | cgggc | 102 | ggcaccgcggggcagcccc | 120 |
| 22 | 25 | 4 m | | 19 n | cgggc | 786 | atgacctcgggcacgctca | 804 |
| 23 | 26 | 4 m | 5 n | 17 n | cgggg | 866 | aatcctgcggggacttcat | 884 |
| 24 | 9 | 4 m | | 5 n | cgggt | 833 | gaagccgcgggtccttcag | 851 |
| 25 | 8 | 3 m | | 5 n | cgggt | 1547 | acgtgaacgggttgctgcc | 1565 |
| 26 | 52 | 3 m | 1 n | 48 n | cggtc | 1654 | tggcccccggtccccccag | 1672 |
| 27 | 7 | 3 m | | 4 n | cggtg | 570 | ttcaagacggtgtatcgag | 588 |
| 28 | 33 | 4 m | | 29 n | cggtg | 735 | tggaagtcggtgctgaggg | 753 |
| 29 | 23 | 3 m | | 20 n | cggtg | 1318 | aggagcgcggtgtgcacgt | 1336 |
| 30 | 292 | 3 m | 10 n | 279 n | gagga | 481 | aagaaaaggaggacatgga | 499 |
| 31 | 153 | 3 m | 15 n | 135 n | attct | 2183 | cgagttcattctgccttcg | 2201 |

Sensitivity and Specificity of SDSO

Although the specificity and sensitivity of an antisense oligonucleotide has been described by those of skill in the art, several related dimensions need further classifying with the establishment of genomic DNA databases and advent of bioinformatics technology. To evaluate the specificity and sensitivity of a selected SDSO relative to the Homo Sapiens database, we applied Matthews correlation coefficient, a measure that is commonly used in bioinformatics, for example in protein structure and gene finding evaluations. This measure can be applied to an efficacious SDSO prediction as well to quantify the agreement between the predicted SDSO and the Human Genome database searches. The sensitivity of a SDSO in the present invention refers to the likelihood that member of a given family has its fully or partially homologous sequence, while the specificity of a SDSO means the likelihood that member of other family has not its fully or partially homologous sequence. Other related terms are defined as follows:

A true positive (TP) is a positive test result obtained for a SDSO in which the member of a given gene family has its full or partial homolog.

A true negative (TN) is a negative test result obtained for a SDSO in which the member of other gene families has not its full or partial homolog

A false positive (FP) is a positive test result obtained for a SDSO in which the member of other families has its full or partial homolog.

A false negative (TN) is a negative test result obtained for a SDSO in which the member of a given gene family has not its full or partial homolog.

In the context of this invention, the sensitivity and specificity of a selected SDSO is related to the length of a sequence, the property of a conserved region, and the types of cleavage pattern in its corresponding genomic RNA sequences. It is well known in the art when the length of a sequence decreases, the probability of this sequence matching its cognate fragment in human genomic sequences will increase. By the way of example, a sequence with the length of 20 nt oligonucleotide will become to match more and more sequences within human genomic RNA molecules with the decrease of base-pairing extent from hundred percent to five percent. In the other word, the sensitivity of this sequence in fishing out its homolog in a human genomic DNA sequence becomes greater and greater, while its specificity will decline. When a conserved sequence can be shared by a given gene family, or by several other gene families, a SDSO homologous to a partial region of this motif can hybridize both the RNA transcribed from that given gene family and other RNA molecules from corresponding gene families. It is true for this sequence to have a higher sensitivity, but it also get a lower specificity. In the dimension of cleavage pattern CGGAU, a higher specificity can be obtained only if all the bases in cleavage pattern CGGAU or GGGAA. Otherwise, a higher sensitivity might occur when other types of cleavage patterns replace them in most cases. Taken together, If the highest specificity is required under the conditions of the invention, the invention recommends that the best condition include but be not limited to that 100 percent of base-pairing between the SDSO and its cognate RNA molecule is complementary to each other, that there is only motif of its homologous RNA in the SDSO, and that the cleavage pattern must be CGGAU or GGGAA in most cases. If the balance between sensitivity and specificity need to meet, the adjustment of these conditions is also easy to reach by using the approaches described in the invention.

The effectiveness of a SDSO in inhibiting the activity of its cognate RNA is the first important issue to any gene therapeutic approaches. It is also closed related to the sensitivity and specificity of a SDSO. However, how to valuate the efficacy of a SDSO was often overlooked in many related patents and scientific papers. The main technological obstacles include that the human genomic projects were just completed, that many genes have not identified, and that bioinformatics technology is going to the benches of biologists. It is well known in the art when a small fragment of oligonucleotide was introduced into a cell, many RNA molecules with its homolog will compete to hybridize it with each other. The more these RNAs exist, the less effective the SDSO will be on a given target RNA. The second cause may be the amount of a given RNA molecule in a cell. The higher the magnitude of the RNA, the lower the effectiveness of the SDSO is. The third is owing to the choice of cleavage site. If a SDSO molecule possesses the strong cleavage site, it will bring the RNase III to its cognate sequence with the strong cleavage site such as CGGAU, and vice versa. The fourth is the extent of base-pairing between target RNA and SDSO. The effectiveness of SDSO decreases with the complementary extent declining. Obviously, the method for enhancing the sensitivity and specificity of a specific SDSO in the present invention benefits to valuate the efficacy of a SDSO and enhance the pharmaceutical effects of selected SDSOs.

Synthesizing, Purifying, Modifying, and Cloning Selected siRNAs

Methods for synthesizing a double-stranded oligonucleotides with a specific sequence pattern are well known in the art. By way of example, a nucleotide sequence can be synthesized chemically by using the solid phase phosphoramidite triester method (Beaucage and Caruthers, 1981, Tetrahedron Letts, 22(20):1859-1862) and an automated synthesizer (Needham-VanDevanter et al. 1984, Nucleic Acids Res., 12:6159-6168). The invention also includes, but is not limited to, double-stranded oligonucleotides made by using the following method.

I. RNA Synthesis

1. 1 mmol G-residue columns (iPr-Pac-G-RNA 500) and oligoribonucleotides (Bz-A-CE Phosphoramidite, U-CE Phosphoramidite, dmf-G-CE Phosphoramidite, and Ac-C-CE Phosphoramidite) with the 2′-O-TBDMS protection (t-Butyl-dimethylsilyl), as well as the RNA synthesis activator (0.25 M 5-Ethylthio-1H-Tetrazole in acetonitrile) from Genset (La Jolla, Calif.) were required for RNA synthesis.

2. Both sense strand (+) and antisense strand (−) of double-stranded oligonucleotides were synthesized using DNA/RNA Synthesizer Model 392 (Applied Biosystems).

| | |
|---|---|
| (+)RNA: | 5′-CCGGGUGCGGAUAAGGGACTT-3′ or DNA |
| (−)RNA: | 5′-GUCCCUUAUCCGCACCCGGTT-3′ or DNA |

3. Modify the coupling time from 10 min to 15 min by setting the synthesis cycle “1.0 mmol RNA” in the machine.

4. It takes about 4 hrs to go through the oligomer synthesis.

II. Cleavage From Support and Removal of Base and Phosphate Protecting Groups

1. Open the synthesis columns and pour the support into a sealable vessel that need not be sterile.

2. Add 1 ml of ethanol/NH₄OH (1:3, v/v) to the vial, seal it tightly and then incubate it at 55° C. for at least 18 hrs.

3. Cool the sealed vial on ice, spin down the support, and open the vial carefully. From now forward, the use of sterile conditions is required. Discard the supernatant, rinse the solid support with 2×1 ml of sterile water, and then combine all solutions.

4. Evaporate the combined solutions to dryness.

III. Removal of 2′-O-silyl Protecting Groups (TBDMS)

1. Add 0.4 ml of tetrabutylammonium fluoride solution (1M in THF) to the residue. Shake the tube gently and leave it at room temperature for at least 6 h.

2. Add 0.4 ml of 1M TEAA solution (aqueous triethylammonium acetate) to the tube, followed by a further 1 ml of sterile water.

IV. Desalting the RNA Oligomers

1. Pour off the azide solution from the desalting column (Bio-Rad Econo-Pac 10 DG) and wash the column with 15 ml of sterile water. Load the RNA solution onto the column, rinse the vial with further 1 ml of sterile water. Collect the eluent. This should not contain any RNA product but keep for now and discard once product isolation is complete.

2. Elute the product from the column with 4 ml of sterile water. Collect this 4 ml eluent that contains the desired product. Further elution with sterile water will yield a small amount of product but it is contaminated with salts.

3. Lyophilize the crude RNA products.

V. RNA Purification by Urea-Acrylamide Gel

1. Prepare a urea-acrylamide gel (7.3 M Urea—20% acrylamid, 16 cm×30 cm).

Urea 70.4 g

10×TBE 16.0 ml

38:2 Stock 80.0 ml

10% APS 1.6 ml

TEMED 60.0 ml

Total volume=160 ml

(38:2 Stock solution—38 g acrylamide+2 g Bis/100 ml)

2. Prepare RNA loading samples.

Dissolve RNA samples in 600 ml (or less) sample buffer (400 ml ddH₂O+100 ml RNA dye buffer+100 ml of 100% glycerol).

Heat samples at 100° C. for 2 min and put on ice immediately.

3. Load samples onto the top of gel and run the gel at 500 V for 2 hr.

4. Cutting RNA bands from the Gel

Put the gel on a TLC plate and check RNA bands using UV light.

Cut the product band using NEW razor blades and slice the gel to small pieces.

5. Extract RNA from the gel.

Soak the small RNA gels in 20 ml of 1×TBE and shake the tubes overnight at 4° C.

Collect the solution and soak the gel pieces in 20 ml of 1×TBE overnight at 4° C. again.

Combine these solutions.

6. Concentrate RNA products.

Add 9 ml of 3 M sodium acetate (final concentration of 0.3 M) and 45 ml of isopropanol (final concentration of 50%).

Keep the solution at −20° C. overnight or −80° C. for 30 min.

Spin down RNAs at 15,000 rpm, 4° C. for 50 min.

Wash RNA pallets with cold 80% EtOH, spin again at 10,000 rpm, 4° C. for 30 min.

Dry the pallets using speed vacuum.

Dissolve these RNAs in 0.5 ml of ddH2O.

7. Desalt the purified RNA oligomers as step 1V, lyophilize and store products at −20° C. The final yield is 1 mg per 1 mmol column.

VI. dsRNA Synthesis

DsRNA is prepared by annealing equimolar concentration of sense RNA/DNA and antisense RNA/DNA in 10 mM Trish (pH 7.5) with 20 mM NaCl (50 ul annealing reaction, 1 uM strand concentration) The reaction mixture is heated at 95 C for 5 min, then gradually cooled down to room temperature, and incubated for 16-20 hrs at room temperature. Most, if not all, single-stranded oligos will converted to double-stranded oligonucleotides.

In one embodiment, the selected and synthesized double-stranded oligonucleotides possess the sequence homologous to a specific segment of RNAs. The functions of corresponding RNAs can be partially influenced or totally blocked in a tumor cell or a pathogenic tissue. By blocking expression of selected genes, cancer growth, viral infection, or genetic disorder can be effectively controlled.

Selecting Appropriate Carriers

Because naked oligonucleotides are poorly incorporated into cells in the PBS fashion, efficient delivery is essential for successful gene drugs of the invention. The delivery system of oligonucleotides includes two classes, which are biological and mechanical ways. The former is composed of viral and nonviral vehicles while the latter comprises manual injection and gene gun. Preferred vehicles of the invention are a complex carrier including but being not limited to cationic liposomes and polymers.

Preferred nonviral classes of compounds include fatty acids and esters, cationic liposomes, cationic porphyrins, fusogenic peptides, and artificial virosomes. These compounds share the characteristic of forming complexes with oligonucleotides through electrostatic interactions between the negatively charged oligonucleotide phosphate groups and positive charges contained by the vehicles themselves. In addition, some degree of protection from nuclease degradation is conferred to the oligonucleotide when associated with such delivery vehicles (De Smedt et al., 2000, Pharmaceutical Research 17:113-126).

Some fatty acids, fatty acid esters, chelating agents and surfactants may be valuable to facilitate the entry of oligonucleotides into cells. Preferred fatty acids and esters include but are not limited 1-dodecylazacycloheptan-2-one, arachidonic acid, caprylic acid, capric acid, dilaurin, diglyceride, dicaprate, eicosanoic acid, glyceryl 1-monocaprate, lauric acid, linoleic acid, linolenic acid, monoglyceride, monoolein, myristic acid, oleic acid, palmitic acid, stearic acid, and tricaprate.

Cationic liposomes are among the most attractive vectors for human gene therapy because they are not infectious and have little immunogenicity or toxicity. Morphologically, cationic liposomes are divided into three main types: small unilamellar vesicles (SUVs), large unilamellar vesicles (LUVs) and multilamellar vesicles (MLVs). Preferred lipids and liposomes include the neutral lipid 1,2-dilauroyl-sn-glycero-3-phosphoethanolamine (DLPE), 1,2-diphytanoyl-sn-glycero-3-phosphoethanolamine (DiPPE) and DOPE that is thought to assist in endosome disruption, and cationic lipid such as dioleoyltetramethylaminopropyl DOTAP and the cytofectin N-[1-(2,3-dioleoyl)phosphatidyl]-N,N,N trimethyl ammonium chloride (DOTMA) as well as N-(α-trimethylammonioacetyl)-didodecyl-D-glutamate chloride (TMAG). Preferred lipid carriers of the invention will generally be a mixture of cationic lipid and neutral lipid at 1:1 ratio.

Alternatives to cationic lipids include cationic porphyrins. Both tetra(4-methylpyridyl) porphyrin (TMP) and tetraanilinium porphyrin (TAP) can more efficiently deliver oligonucleotides into cells than naked oligonucleotides. Moreover, cationic porphyrins not only help oligonucleotides delivery into the cell, but they are also able to localize the oligonucleotides in the nucleus where mRNA and RNase III are present.

Artificial virosomes are another class of delivery vectors which take advantage of the natural ability of a virus to gain entry into cells. Reconstituted influenza virus envelopes known as virosomes can fuse with endosomal membranes after internalization through receptor-mediated endocytosis. Recently, cationic lipids have been incorporated into virosome membranes to further aid delivery.

The polycationic agents are another useful means to enhance cationic liposome-mediated entry. Preferred cationic polymers include poly-L-lysine(pLL), procaine sulfate (PA), recombinant human HI his tone protein, sperm dine and polyethylenimine (PEI). PEI has been shown to be an efficient nonviral vehicle for gene delivery to a variety of cells, and to promote oligonucleotide location to the nucleus in mammalian cells. The distinctive characteristics of PEI such as nucleic acid-binding and condensation, along with its high buffering capacity and intrinsic endosomolytic activity is considered to protect nucleic acids from degradation. High reporter gene expression was found with complexes using the linear 22 kDa PEI in topical and systematic application. Despite the similar in vitro transfection behavior of all forms of PEI, in vivo branched 25 kDa PEI proved superior to linear 22 kDa PEI. When these properties of PEI were combined with the specific mechanism of receptor-mediated gene delivery, ligand-conjugated PEI resulted in higher transfection efficiency in various tumor cell lines (O'Neil et al., 2001, Gene Therapy 8:362-368).

Fusogenic peptides form peptide cages around oligonucleotides in order to boost oligonucleotide uptake. Many of these peptides contain polylysine residues, which cause membrane destabilization. Generally, these agents are less cytotoxic than lipids but are still able to achieve similar delivery efficacy.

Except for old manual injection, the recently developed “gene gun” device employed DNA-coated gold particles that are accelerated by pressurized helium gas to supersonic velocity for DNA transfer into living cells.

Selecting Specific Cell-Targeting Molecules

An important topic of gene drug is to deliver (tissue targeting) a therapeutic gene drug to target cells or tissues, without affecting healthy cells or tissues. Tissue targeting can be accomplished by direct intra-tissue injection of the gene drug or with cell- and tissue-aiming molecules such as antibodies, ligands, or viral particles. Many methods have been introduced in the art.

Specific targeting systems of the invention prefers include but are not limited to the following major dimensions:

1. targeting antibodies with the following examples;

high-affinity monoclonal antibodies, AF-20 which recognizes a rapidly internalized 180 kDa cell surface glycoprotein was used to facilitate gene transfer to hepatic cancer cells.

an anti-CD3 antibody conjugated to poly-L-lysine was used to facilitate gene transfer via the CD3 receptor in primary lymphocytes for the treatment of related leukemia.

immunoconjugated liposomes labeled with human single chain fragment of variable region of anti-high molecular weight-melanoma associated antigen antibody (HMW-MAA) can be employed to target the gene to metastasis lesions.

2. targeting carbohydrate or protein ligands as follows;

glycoprotein specific for the receptors present on CD4-positive T cell used for gene delivery to human T cells, which can be used in treating AIDS or T cell leukemia,

cholesteryl-spermidine employed for highly specific and efficient non-viral target gene delivery to AF-20-positive cells in hepatoma,

adenovirus specific for the CAR receptor (receptor for retrovirus and coxacki virus) on related cells such as lung cancer cell,

a high-efficiency nucleic acid delivery system based on transferrin receptor-mediated endocytosis, which carries DNA into related cells.

A combination of stearyl-polylysine, low-density lipoprotein (LDL) and nucleic acid targeted to a desired location through the specific LDL receptors in obesity patients.

3. targeting means:

a new system for the generation of Penetratin coupled polypeptides with the potential for both in vitro and in vivo gene targeting developed by Qbiogene. The 16 amino acid long peptide, Penetratin, corresponds to the DNA binding domain. It has the ability to translocate hydrophilic oligonucleotides to the cytoplasm and nucleus of living cells.

Other Ingredients

The compositions of the present invention may contain other adjunct components as conventional medicine does. The compositions may include but be not limited to:

anti-inflammatory agents such as nonsteroidal anti-inflammatory drugs and corticosteroids,

antioxidants,

dyes,

flavoring agents,

gels

local anesthetics,

lubricants,

preservatives,

stabilizers,

thickening agents,

wetting agents,

However, these materials, when added, should not influence the biological function of siRNAs of the compositions of the present invention.

Assembly of Gene Drug

The assembly of a gene drug is related to many issues including the proportion of double-stranded oligonucleotides to lipids, their concentrations, pH value of the buffer, ionic strength and other stability-enhancing reagents. The main issues examined were In order to avoid or reduce complex precipitation, to protect double-stranded oligonucleotides from degradation mediated by a nuclease, and to enhance transfection efficiency, the formulation of compounds or compositions in the invention comprise the following preferred conditions for transfection:

5% (w/v) dextrose in 10 mM PBS (pH 6.5),

low ionic strength solutions (double steamed water and 60% ethanol w/w),

1:6 ratio for double-stranded oligonucleotides vie lipid

components of lipid:phosphatidylcholine and phosphatidylserine,

pH value at 6.5

concentration of double-stranded oligonucleotides: 0.4-1 ug/ul

carriers' size

In addition to the conditions mentioned above, preferred mean transfection complex size for topic administration is from 30 to 60 nm. Preferred mean transfection complex size for aerosol administration is from 50 to 200 nm. Preferred mean transfection complex size for intravenous administration is from 200 to 600 nm.

Active ingredients: groups of different specific siRNAs that can efficiently suppress their corresponding target RNAs. According to abnormal over-expression of a group of genes in different diseases, types of siRNAs and their combination will be adjusted in order to achieve the maximal therapeutic ends and minimal advert effects.

Double-stranded oligonucleotides (2 ul) and cationic liposomes (6 ul) were placed at the bottom of a 7 ml sterile Bijou container, but not in contact with each other. RNA and liposomes were combined by the addition of 42 ul serum-free differentiation media and gentle shaking. Lipoplex mixtures were then incubated at room temperature for 20 to 30 min before being applied to cells. Lipopolyplex mixtures were generated in the following manner. 25 kDa branched PEE (2 ul) was placed in the bottom of sterile polystyrene containers alongside, but not in contact with siRNA(2 u.I) and mixed by the introduction of 40 ul of 150 mM NaCl. These polyplex mixtures were then incubated at room temperature for 10 min after which time the mixture of neutral lipid DOTMA and cationic lipid DOPE (6 uI) were added. Resulting lipopolyplex mixtures were then further incubated at room temperature for 20 min before being applied to cells.

The Characteristics of Gene Drug

Since a drug is defined as any chemical agent that regulates the process of living, the gene drug is one of chemical agents, which affects the functions of living cell in the form of oligonucleotides.

Characteristics of Gene Drug

A gene drug should posses the following characteristics:

1. the failure to change the genetic information of any normal genes,

2. the interaction with specific segment of DNA, target mRNA or any other aimed RNA molecule that is one disease-causing factor,

3. and the interference, reduction or removal of the syntheses of corresponding peptide or protein,

Structure of Active Ingredients of Gene Drugs

Most preferred embodiments of the invention are 21 nt double-stranded RNA with 5*-phosphatey3*-hydroxyl ends and a 2-base 3* overhang on each strand of the duplex, with one cleavage pattern CGGAU in its center. Also preferred are other types of SDSO such as 19-25 nt sRNA-cDNA and dsDNA having one cleavage pattern CGGAU or its derivatives including but being not limited to CGGAA, CGGAC, CGGAG, CGGGA, CGGGU, or CGGGC.

Short interfering RNAs (siRNAs) are double-stranded RNAs of 21 nucleosides that have been shown to play key roles in triggering sequence-specific mRNA degradation during posttranscriptional gene silencing in plants and RNA interference in animals and human beings. The basic structure of SDSO is shown in the following tables 5, 6, and 7. Each of the SDSOs indicated in Table 2 that inhibited expression of a gene comprised a CGGAT or CGGGA cleavage pattern was homologous to a region of an mRNA molecule encoding a protein. All the evidence proves that a RNA-based SDSO can be designed by selecting a SDSO including a CGGAT, CGGGA or their derivatives. Although RNA-based SDSOs comprising 19 nucleotide residues in each strand have been described herein, it is clear, given the data presented herein, that other types of SDSOs may be designed which comprise 19 to 25 nucleotide residues including a specific cleavage center. Preferably, such SDSOs start at a letter A or one of T(U), C, G following the letter A in the same genomic DNA sequence, and end at a letter T, comprising all nucleotide residue which is completely homologous to their genomic DNA encoding corresponding RNA molecules. The ability of these SDSOs to suppress expression of a gene may be easily assessed by employing the simplified selection methods described herein.

[image]

The Compounds of Gene Drugs

The Kind of Double-Stranded Oligonucleotides

In one embodiment of the present invention, the compositions of oligonucleotides are formulated as a mixture, which may include different kinds of double-stranded oligonucleotides such as 19-25 nt dsRNA, sRNA-cDNA, or dsDNA shown in Table 5, 6, and 7. The different compounds of these three oligonucleotides may bring out different long-term and short-term therapeutic effects (Table 8) as conventionally pharmaceutical agents did. They may play other biological functions such as the methylation of DNA, the spread of silencing signal, and self-amplification of siRNA molecule.

TABLE 8
Different kinds of double-stranded oligonucleotides and their functions.

| | siRNA | sRNA-cDNA | siDNA |
|---|---|---|---|
| Short-term eff. | Antisense RNA | cDNA | Antisense |
| | | | DNA |
| Long-term eff. | Sense RNA | Sense RNA | None |
| Target enzyme | RNase III, Helixase, | RNase H, Helixase? | RNase H, |
| | | | Helixase? |
| Self synthesis | RNA polymerase II? | RNA polymerase II? | |
| DNA Methyl. | Methyltransferase | Methyltransferase? | |

One or More Double-Stranded Oligonucleotides

In another related embodiment, the active ingredients of the composition of the invention may include one or more different types of double-stranded oligonucleotides, particularly the first oligonucleotides aimed to a first nucleic acid, and the second or the nth additional antisense compounds targeted to a second target mRNA, or a nth target mRNA. This way that combines many different active agents together for a specific therapeutic aim is well known in the art. Two or more combined double-stranded oligonucleotides may be used together or sequentially. In the following context, the compounds of gene drugs will be described in details.

Different Dose of the Same Double-Stranded Oligonucleotides

One, two, or three different kinds of double-stranded oligonucleotides, different dose of the same agent, or any combination thereof.

The Forms of Gene Drugs

The gene drugs can be delivered in a variety of forms. They are:

transdermal patches,

ointments,

lotions,

creams,

drops,

sprays,

liquids

powders

Conventional pharmaceutical carriers, aqueous, powder or oily bases, thickeners and the like may be necessary or desirable.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable.

The Delivery of Gene Drugs

The pharmaceutical compositions and formulations of the present invention include 19-25 nt dsRNA, sRNA-cDNA or dsDNA. In addition to double-stranded oligonucleotides, such pharmaceutical compositions may include pharmaceutically acceptable carriers and other ingredients known to enhance and facilitate drug administration. The active medicine ingredients of the present invention may be administered in the following ways:

topical delivery including ophthalmic, vaginal and rectal supplement,

inhalation or insufflation of powders or aerosols including intratracheal, intranasal, epidermal and transdermal use,

oral or parenteral administration including intravenous, intraarterial, subcutaneous, intraperitoneal or intramuscular injection or infusion,

intracranial delivery including intrathecal or intraventricular administration.

A type of gene drug of the invention may be delivered by following another one or other therapeutic means.

The Usage of Gene Drugs

The formulation of therapeutic compounds and their subsequent administration is believed to be well known in the art. Dosing is dependent on severity and responsiveness of the disease state to be treated and conditions of the patient health, with the course of treatment lasting from several days to several months, or until a cure is reached or a diminution of the disease state is achieved. Optimal dosing schedules can be calculated from measurements of drug accumulation in the body of the patient. Professional persons can easily determine optimum dosages, dosing methodologies and repetition rates. Optimum dosages may vary depending on the relative potency of individual oligonucleotides, and can generally be estimated based on EC50S found to be effective in vitro and in vivo animal models. In general, dosage is from 5 ng to 200 mg per kg of body weight, and may be given once or more daily, weekly, monthly or yearly. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotides are administered in maintenance doses, ranging from 5 ng to 200 mg per kg of body weight, once or more daily, weekly, monthly or yearly.

Metabolic Mechanisms of Gene Drugs

Mechanisms that silence unwanted gene expression are critical for normal cellular function. Gene silencing mechanisms include a variety of transcriptional and posttranscriptional surveillance processes. Double-stranded RNA (dsRNA) has been reported to induce at least four posttranscriptional surveillance processes.

The first major pathway of the nonspecific response to dsRNA is mediated by the dsRNA-dependent protein kinase (PKR), which phosphorylates and inactivates the translation factor eIF2a, leading to a nonspecific suppression of all protein synthesis and cell death via both nonapoptotic and apoptotic pathways. dsRNA can activate PKR in the length-dependent manner. dsRNAs of less than 30 nucleotides are unable to switch the transforming of PKR, while more than 80 nucleotides can fully activate PKT.

The second one is related to 2-5A-dependent RNase L pathway. It has also been demonstrated that a second dsRNA-response pathway involves the dsRNA-induced synthesis of 2′-5′ A polyadenylic acid and a consequent activation of a sequence-nonspecific RNase (RNaseL).

The third one is concerned with the RNAi. A long dsRNA can be broken into many short dsRNA mediated by a RNase III. The resulting siRNAs can silence their cognate gene involving the degradation of single-stranded RNA (ssRNA) targets complementary to the dsRNA trigger. Similarly, the RNAi employed by the normal cells to inactivate some mRNAs may be a very effective approach against aberrant genomic attack in which there exist the over expression of genes, abnormal functions and structures of genes, and invaded genetic elements such as virus, bacteria, and fungi. Taken together, RNAi is a set of natural defensive mechanisms in cells of the living organisms.

The fourth way is formed by the derivatives of the pathways mentioned above or aberrant single-stranded RNA or DNA molecules, which can initiate a typical antisense pathway mediated by a RNase H or other nucleases. However, this pathway is different from that way mediated by introducing a single-stranded cDNA. A single-stranded cDNA or ssRNA antisense oligonucleotides require the extensive chemical modifications to enhance the in vivo half-life. It will enhance the cost and other side effects. However, the ssRNA or cDNA produced by introducing a SDSO has a longer half-life because it has an opportunity to form a duplex with its another half in a cell.

Recently, several lines of evidence indicated that the interference by 21-25 nt double-stranded oligonucleotides were superior to the inhibition of gene expression mediated by single-stranded antisense oligonucleotides. The siRNAs seem to avoid the well-documented nonspecific effects triggered by longer double-stranded RNAs in mammalian cells. Moreover, many studies have demonstrated that siRNAs seem to be very stable and thus may not require the extensive chemical modifications. More importantly, the siRNAs are able to produce specific inhibition in expression of target genes.

After the comparison of the antisense and RNAi technology conducted by several laboratories, it was indicated that the ssRNA antisense oligomers just partially inhibited expression of a gene while the siRNA-mediated inhibition was more potent (′1.5-fold). The results suggested that the gene silencing mediated by the small dsRNAs can be distinguished from a purely antisense-based mechanism. Obviously, These observations may open a path toward the use of 21-25 nt double-stranded oligonucleotides as a reverse genetic and therapeutic tool in human.

Furthermore, 19-25 nt double-stranded oligonucleotides have been found to involve in the methylation process of genomic DNA. DNA methylation cannot only suppress the expression of genes, and also increase the probability that affected genes undergo a mutational event. Although DNA methylation plays a key role in normal biologic processes, its abnormal patterns of methylation result in cancers. In particular, several lines of evidence demonstrated that methylation within the promoter regions of tumor suppressor genes such as P53 and Rb causes their silencing, and methylation within the encoding gene itself can induce mutational proteins. All this constitutes both the important molecular basis of a cancer development, and the therapeutic barrier to many current treatment. A brand-new treatment idea from this invention is that siRNAs are very good counter forces to the cancer genesis because the siRNAs are implicated as the guides for both a nuclease complex that degrades the mutant mRNA and a methyltransferase complex that methylates the DNA of diseased genes. Thus, the new balance in the methylation and expression between diseased and normal genes will be reached again in the cancer cells, and finally, the malignance of cancer cell will go down to nothing. In addition, a SDSO molecule can be designed to inhibit the gene encoding a methyltransferase specific for methylating the promoter regions of tumor suppressor genes.

### EXAMPLE-1

### Evaluation of the Specificity of SDSO Molecule Selected by Simplified Method

The table 9 demonstrated that the sequences predicted by simplified method possess high specificity and efficiency of cleavage. In the homo sapiens c-myc proto-oncogene, there are five different regions that contain the cleavage sequence patterns. When these sequence with 19 nucleotides were used as the query sequence, they all displayed much better specificity than sequences with other cleavage patterns in the center of their sequences. For example, sequence 2, 3, 4, 5, 6, in seq.ID#5 got pretty specific hits, while a random selection of two sequences from the c-myc gene will cause a serious problem in specificity. These two sequences fished out high hits of homologous sequences such as sequences 1 and 7 in seq.ID#5.

TABLE 9
gi|11493193: Homo sapiens MYC gene for c-myc proto-oncogene and ORF1

| Seq. | Total | 100% | 80-95% | <80% | | Start | | End |
|---|---|---|---|---|---|---|---|---|
| ID#5 | Hits | Match | Match | Match | Pattern | Point | Sequence | Point |
| 1 | 118 | 19 m 3 n | 1 n | 94 n 1 m | aggaa | 21 | caccaacagg aactatgacc | 39 |
| 2 | 29 | 17 m 2 n | 1 n | 9 n | cggaa | 1296 | acagc tacggaactc ttgt | 1314 |
| 3 | 34 | 15 m 3 n | | 16 n | cggaa | 1254 | cttgttg cggaaacgac ga | 1272 |
| 4 | 41 | 16 m 3 n | | 22 n | cggaa | 939 | ct ccactcggaa ggactat | 957 |
| 5 | 39 | 15 m 3 n | | 21 n | cggag | 1107 | gcta aaacggagct ttttt | 1125 |
| 6 | 24 | 17 m 3 n | | 4 n | cggac | 349 | tg cgacccggacgacgaga | 367 |
| 7 | 217 | 18 m 3 n | | 196 n | ccgcc | 541 | ctgagcgccg ccgcctcag | 559 |

The table 10 listed the searching results of different 21 nt portions of a mdm2 gene. Four 21 nt sequences fished out high hits of homologs although one of them could get pretty specific hits, suggesting that a random selection of a sequence from the given gene will cause a serious problem in specificity, and needs more trials in order to get higher specificity. On the other hand, when a sequence with a specific cleavage pattern is selected, it will obtain very specific hits.

TABLE 10
XM_052466 GI:14762555: Homo sapiens similar to mouse double minute 2, human
homolog of p53-binding protein (H. sapiens) (LOC113222), mRNA.

| Seq. | Total | 100% | 80-95% | <80% | | Start | | End |
|---|---|---|---|---|---|---|---|---|
| ID#6 | Hits | Match | Match | Match | Pattern | Point | Sequence | Point |
| 1 | 52 | 31 m | | 21 n | cggaa | 58 | ccagcttcggaac aagaga | 76 |
| 2 | 135 | 35 m | 3 n | 97 n | aactt | 371 | ttgtgctaac ttatttccc | 389 |
| 3 | 302 | 34 m | 11 n | 257 n | gtgca | 301 | tttacatgtg caaagaagc | 319 |
| 4 | 111 | 32 m | 1 m | 78 n | gtctg | 11 | ccaacatgtc tgtacctac | 29 |
| 5 | 39 | 31 m | | 8 n | gacct | 241 | caaggtcgac ctaaaaatg | 259 |
| 6 | 347 | 33 m | 17 n | 307 n | agaaa | 161 | aaagggaaga aacccaaga | 179 |

The table 11 shows another example for the importance of cleavage patterns in predicting an efficacious SDSO. Comparison of the results obtained by the CGGAT pattern and other patterns in selecting a portion of a TGF-beta2 gene as aSDSO demonstrated that the CGGAT pattern had much better prediction than other patterns did.

TABLE 11
gi|31959: transforming growth factor-beta2, TGF-beta2

| Seq. | Total | 100% | 80-95% | <80% | | Start | | End |
|---|---|---|---|---|---|---|---|---|
| ID#7 | Hits | Match | Match | Match | Pattern | Point | Sequence | Point |
| 1 | 193 | 6 m | 25 n | 162 n | ctgat | 31 | cgcttttctg atcctgcat | 49 |
| 2 | 196 | 5 m | 7 n | 184 n | tttct | 1201 | gaacagcttt ctaatatgat | 1219 |
| 3 | 12 | 5 m | 1 n | 6 n | cggat | 486 | tgaac aacggattga gcta | 504 |
| 4 | 106 | 5 m | 2 n | 99 n | gggat | 976 | ttcaa gagggatcta gggt | 994 |
| 5 | 112 | 6 m 1 n | 13 n | 92 n | agatc | 121 | cgcgggcaga tcctgagca | 139 |
| 6 | 211 | 7 m | 85 n | 109 n | ccctt | 321 | catgccgccc ttcttcccct | 339 |
| 7 | 241 | 5 m | 14 n | 222 n | gggaa | 819 | aa acagtgggaa gacccca | 837 |

The table 12 compared the specificity of different sequences located in Homo sapiens telomerase RNA gene. The sequences predicted by the simplified method have lower hits and less homologous to the sequences derived from other gene families. The sequence 4 in SeqID#8 is the best one that starts at A and has two strong cleavage sites.

TABLE 12
AF221907: Homo sapiens telomerase RNA gene, sequence

| Seq. | Total | 100% | 80-95% | <80% | | Start | | End |
|---|---|---|---|---|---|---|---|---|
| ID#8 | Hits | Match | Match | Match | Pattern | Point | Sequence | Point |
| 1 | 54 | 2 m | 1 n 1 m | 48 n 2 m | gactc | 1 | agagagtgac tctcacgag | 19 |
| 2 | 20 | 4 m | | 16 n | cggaa | 223 | cagcgggc ggaaaagcctc | 241 |
| 3 | 67 | 4 m | 4 n | 59 n | cagga | 521 | gtgcacccag gactcggct | 539 |
| 4 | 12 | 4 m | 1 n | 8 n | cggag | 469 | ag aggaacggag cgagtcc | 487 |
| 5 | 528 | 4 m 1 n | 25 n | 499 n | gggag | 111 | tgggcctggg aggggtggt | 129 |
| 6 | 66 | 3 m 1 n | 3 n | 59 n | ccgaa | 327 | ccag cccccgaacc ccgcc | 345 |

In the table 13, two cases should be paid attention to. That is Sequences 2 and 5 in SeqIld#9, which suggested that some sequences without the special cleavage pattern could also have high specificity. However, the problem about cleavage strength remains even although those sequences contain weak cleavage sites. At least, the efficiency of cleavage mediated by RNase III should be influenced.

TABLE 13
gi|10863872: Homo sapiens transforming growth factor, beta 1 (TGFB1)

| Seq. | Total | 100% | 80-95% | <80% | | Start | | End |
|---|---|---|---|---|---|---|---|---|
| ID#9 | Hits | Match | Match | Match | Pattern | Point | Sequence | Point |
| 1 | 72 | 6 m 1 n | 2 n | 63 n | cctcc | 1 | atgccgccct ccgggctgc | 9 |
| 2 | 22 | 7 m 1 n | | 14 n | tgatc | 1141 | tccaacatga tcgtgcgctc | 1159 |
| 3 | 18 | 8 m 1 n | | 9 n | cggag | 599 | at gtcaccggag ttgtgcg | 617 |
| 4 | 50 | 7 m 1 n | 8 n | 34 n | cggag | 767 | gcagaaccggagcc cgagc | 785 |
| 5 | 46 | 8 m 1 n | 1 n | 36 n | tccgc | 901 | attgacttcc gcaaggacct | 929 |
| 6 | 319 | 8 m 1 n | 14 n | 296 n | tgttc | 391 | atatatatgt tcttcaaca | 409 |
| 7 | 244 | 7 m 1 n | 28 n | 208 n | gggga | 189 | ga gccagggggaggtgccg | 207 |

The table 14 indicated that although the simplified method can selected sequences with both high specificity and efficiency of cleavage, there is difference in specificity among those sequences selected. However, by comparison with these sequences, the best sequence will be obtained such as the sequence 4 in SeqID#10.

TABLE 14
gi|14759971: Homo sapiens cyclin-dependent kinase 2 (CDK2)

| Seq. | Total | 100% | 80-95% | <80% | | Start | | End |
|---|---|---|---|---|---|---|---|---|
| ID#10 | Hits | Match | Match | Match | Pattern | Point | Sequence | Point |
| 1 | 51 | 10 m | 3 m 5 n | 33 n | cggag | 23 | aaaagatc ggagagggcac | 41 |
| 2 | 53 | 10 m | | 43 n | caagc | 761 | atgtgaccaa gccagtacc | 779 |
| 3 | 27 | 10 m | 1 n | 16 n | cggac | 540 | catctttcgga ctctgggg | 558 |
| 4 | 20 | 9 m | | 10 n 1 m | cgggc | 489 | ga ctcgccgggc cctattc | 507 |
| 5 | 503 | 10 m | 90 n | 403 n | cagct | 321 | tctgttccag ctgctccag | 339 |
| 6 | 150 | 10 m | 3 n | 137 n | tgcac | 241 | gaatttctgc accaagatc | 259 |
| 7 | 77 | 10 m | 1 n | 66 n | ggagc | 161 | tgcttaagga gcttaacca | 179 |

The table 5 gave another example which proved the usefulness of the simplified method. The sequence 4 in SeqID#11 predicted by the simplified method displayed a higher specificity compared to other sequences selected by the random selection way.

TABLE 15
gi|14750937: Homo HGF

| Seq. | Total | 100% | | 80-95% | <80% | | Start | | End |
|---|---|---|---|---|---|---|---|---|---|
| ID#11 | Hits | Match | | Match | Match | Pattern | Point | Sequence | Point |
| 1 | 359 | 17m | 2n | 17n | 326n | cctgc | 11 | ccaaactcctgccagccct | 19 |
| 2 | 87 | 16m | 2n | | 69n | gggat | 697 | cagc gctgggatca tcaga | 716 |
| 3 | 139 | 13m | 2n | 1n | 126n | cttgc | 1381 | tgggattatt gccctattt | 1399 |
| 4 | 43 | 12m | 2n | 1n | 28n | cggaa | 1655 | atgtccacggaagaggaga | 1673 |
| 5 | 81 | 12m | 2n | 1n | 66n | taagg | 2161 | ttaacatata aggtaccac | 2179 |
| 6 | 90 | 17m | 2n | 2n | 69n | gggaa | 403 | gctacaa gggaacagta tc | 422 |

These are stability, ability to be targeted to the cell of interest, ability to achieve sufficient intracellular concentration to cleave to the targeted mRNA, ability to hybridize with their mRNA target, and lack of toxicity.

The compounds of the invention can be utilized in pharmaceutical compositions by adding one or more effective amount of SDSO compound to a suitable pharmaceutically acceptable diluent or carrier. Use of the SDSO compounds and methods of the invention may also be useful prophylactically, e.g., to prevent or delay infection, inflammation or tumor formation.

### EXAMPLE-2

### Three Groups of Experiments Read as Follows

In vitro cells cultures: The human melanoma cell lines A375 were obtained from the American Tissue Type Culture Collection (ATCC). Melanoma cell lines MC 66 were a kind gift from Dr. Wan (Providence College, RI); All cell lines were maintained in Dulbecco's modified Eagle's culture medium (DMEM, 4.5 g/l glucose), supplemented with 8% fetal bovine serum, 100 units/ml penicillin, 100 ug/ml streptomycin and 0.25 μg/ml amphotericin B (Gibco BRL). For this experiment, 1 ml of melanoma cell suspension in culture medium (2×10⁴/ml) was placed in each well of a Falcon plate (047, Franklin Lakes, N.J., USA) and incubated at 37° C. for 24 h in a humidified atmosphere of 5% CO₂. The culture medium and cells was collected 1, 2, 3, 4, 5 and 6 days respectively after addition of the mixture of serum-free media, liposome or Fugene, and Dermogene (shown in Example 4) according to the manual of Fugene Inc. and The growth-inhibitory effect of Dermogene transfer to melanoma cells was evaluated by an automatic counter, and the amount of corresponding RNAs were measured.

Animals

Female nude mice, KSN, aged 6-8 weeks, were used. They were kept and bred under pathogen-free conditions in the animal facility.

Fragments of the tumors (3 mm in diameter) were transplanted subcutaneously onto the backs of mice by means of a trocar needle. When the transplanted tumors had grown to 7 mm in diameter, the mice were divided randomly into the following four treatment groups: group 1, intratumoral injection of PBS (30 ul) every day; group 2, intratumoral injection of 30 ul empty liposome in the way of one injection every day; group 3, intratumoral injection of 30 ul liposome containing 5 ug Dermogene every other day; group 4, intratumoral injection of 1 mg cyclophosphamide and 30 ul every other day; and group 5, intratumoral injections of 30 ul liposome containing 5 ug of the mixture of Dermogene and 1 mg cyclophosphamide every day. In all the groups, the liposome was injected with a 30-gauge needle every day. The needle was withdrawn after 10 seconds. Growth inhibition of transplanted tumours was evaluated by measuring the tumour size every 2 days with the aid of microcallipers. Tumor volume was calculated using the formula ab²/2, where a is the width and b the length of the tumor. The relative tumor size (%) was calculated from the formula Tₙ/T₀×100, where T₀=tumor weight immediately before the intratumoral injections and Tₙ=tumor weight after the injections.

### EXPERIMENT 1

Viable cultured melanoma cells were counted 1, 2, 3 and 4 days after the administration of Dermogene (FIGS. 9 and 10). Growth inhibition can be observed in both human melanoma cell lines. The growth-inhibitory effects were correlated with the level of Dermogene in the culture medium. Adding 1 ul liposome with 100 ng/ml of Dermogene to the medium of MC66 cells caused an detectable level of cancer cell death, and the growth-inhibitory effects were increased significantly when the dose of Dermogene increased from 5 ng/ml to 500 ng/ml (data not shown in here). No further increase in cancer cell death was observed with the dose over 500 ng/ml. Treatment with empty liposomes did not affect cell growth in any of the cell lines.

### EXPERIMENT 2

In the vivo experiment, tumors injected with PBS every other day grew linearly from the time of injection to a volume two and half times the size by 35 days after the implantation (FIG. 11). In contrast, every other day injections of liposomes containing Dermogene (group 3) and injections of 1 mg Cyclophosphamide and 200 nmol lipid suppressed tumour in its implanted size for 35 days and inhibited tumor size by 40-80% at 35 days after the implantation into a mouse. Surprisingly, administration of 1 mg Cyclophosphamide and 200 nmol lipid every other day can inhibit the growth of tumor for fifteen days, and then loss its ability to suppress the proliferation of tumor cells. No growth inhibition was observed in tumors receiving injection of empty liposomes (group 2) every other day. In mice receiving every day intratumoral injections of liposomes with Dermogene and Cyclophosphamide (group 5) the size of the tumors was suppressed and the tumors disappeared completely within 35 days post-implantation.

### EXPERIMENT 3

### 21 nt siRNAs Block Proliferation and Survival of Primary CML Cells

The CML cells from patients containing a bcr/abl gene were maintained in RPMI 1640 medium (GIBCO-BRL, Gaithersburg, Md.). Primary cells were isolated from bone marrow of three CML patients in chronic phase by Ficoll-Hypaque density gradient sedimentation.

To determine the effect of 21 nt siRNAs on the growth and survival of primary, leukemia cells, bone marrow aspirates from three CML patients were analyzed. Chromosome analysis was performed on 30 cells from each of the three patients' bone marrow. Bone marrow cells of the three patients were cultured and then treated with the SDSOs. In every case, treatments of 100 ng/ml of Leukogene (shown in Example 4) against bcr and abl mRNAs, BCL6 and N-ras caused cell proliferation to cease after 24 hours (FIG. 12). The Leukogene in the dose of 100 ng/ml with 200 nmol lipid can efficiently inhibit the proliferation of CML cells derived from (CML1) patient 1, (CML2) patient 2, and (CML3) patient 3, while empty liposome without any active SDSO molecules failed to suppress the growth of CML cells as shown in CMLC-1, CMLC-2 and CMLC-3.

### EXAMPLE 3

### Analyzing Reported Efficacious SDSOs by Blast Sequence Alignment

To identify efficacious SDSOs that had been reported in other laboratories, A comprehensive search was conducted using the Pubmed database, current through August 2000. These sequences were examined to determine whether a higher proportion of the sequences were characterized with a 100% of homolog to most members of corresponding gene family and minimal similarity to other sequences derived from other gene families.

For the literature search, ASOs selected from among many ASOs include both effective and ineffective sequences that can target a broad range of RNA regions. ASOs present in FDA-approved human clinical trials and related patents were also included in the search. In the table 16, sets of ASOs with different effectiveness on expression of related RNA were employed to evaluate the quality of SDSO molecules that the invention predicted and selected. Five sequences with high effects on inhibiting the expression of WWP2 mRNA was detected by Blast multiple alignment. The results demonstrated that all the five sequence identified have less hits with more 100% of matches to members' of the same gene family and less similarity shared by other sequences. The sequence High5 was the best one that can fish out most of members of its family without any similarity shared by other genomic sequences. All these five sequence can inhibit the activity of corresponding mRNA by more than 80%. On the other hand, it was indicated that four sequences with the inhibiting rate at less than 20% displayed much low specificity with more similarity to other sequences at a wide range from 50% to 95%. More importantly, a group of sequences with specific cleavage pattern were found to be as good as the high group in multiple sequence alignment, compared to bad alignment in the Low group. The nucleotide sequences of the most effective known SDSOs comprising the specific cleavage pattern are listed in Table 16. By comparison, a sequence with other patterns has more chance to show a low specificity with more hits at low matches. Thus, it appears that the specific cleavage pattern can be an excellent indication for selecting a genomic DNA sequence as a target portion of corresponding RNA for an efficacious SDSO molecule.

TABLE 16
XM_028151.2 GI:15318611: Homo sapiens Nedd-4-like
ubiquitin-protein ligase (WWP2), mRNA.

| Seq. | Total | 100% | | 80-95% | | <80% | | Cleav. | Start | | End |
|---|---|---|---|---|---|---|---|---|---|---|---|
| ID | Hit | Match | | Match | | Match | | Pattern | Point | Sequence | Point |
| High1 | 16 | 6m | 1n | | | 9n | | cggt | 54 | cttcacggtgatgatatgg | 72 |
| High2 | 39 | 6m | 1n | | | 32n | | cggt | 52 | agcttcacggtgatgatat | 70 |
| High3 | 24 | 5m | 1n | 1n | | 17n | | cggt | 50 | cagcttcacggtgatgatat | 69 |
| High4 | 14 | 6m | 1n | | | 7n | | | 142 | gtgtccgcaa agcccaaggt | 160 |
| High5 | 7 | 7m | | | | | | | 173 | acctcgaa ttaactccta c | 191 |
| Low1 | 93 | 5m | | 12n | | 76n | | | 2800 | tggtcccacacagggccaca | 2781 |
| Low2 | 123 | 2m | | 26n | | 97n | | | 1360 | cattgtcctgtcttttctcc | 1341 |
| Low3 | 59 | 3m | | 18n | | 38n | | ggga | 1961 | tgtagaaagggagggtgaag | 1942 |
| Low4 | 84 | 3m | | 25n | | 56n | | | 530 | aggaaaattgtcagttttcc | 511 |
| Med | 59 | 6m | 1n | 14n | | 38n | | | 917 | ttcctctccttcagccggtg | 898 |
| Med | 25 | 4m | 1n | 10n | | 10n | | | 1035 | tattgtggtcaacataatag | 1016 |
| Med | 28 | 2m | | 8n | 1m | 17n | | | 1239 | aggaatctttggctgaag | 1222 |
| CGG1 | 15 | 6m | 1n | | | 7n | | cggac | 635 | aagatcccggacgcacaga | 653 |
| CGG2 | 47 | 6m | 1n | 1n | | 39n | | cggag | 435 | ctgcagacggagaacaaag | 453 |
| CGG3 | 56 | 3m | 1n | 1n | | 51n | | cggag | 463 | tctcaggcggagagctgac | 481 |
| CGG4 | 22 | 6m | 1n | | | 15n | | cggag | 704 | cggtgctcggagccggcac | 722 |
| CGG5 | 10 | 6m | 1n | | | 3n | | cgggt | 921 | agcacttcgggtacacagc | 939 |
| CGG6 | 6 | 4m | 1n | | | 2n | | cggac | 1000 | tgcccaacggacgtgtcta | 1018 |
| CGG7 | 31 | 3m | | | | 28n | | cgggc | 1931 | atcgacacgggcttcaccc | 1949 |
| CGG8 | 16 | 3m | | | | 13n | | cggat | 1957 | ctacaagcggatgctcaat | 1975 |
| CGG9 | 51 | 1m | | 1n | | 47n | 2m | cgggt | 2143 | gagcatccgggtcacagag | 2161 |
| CGG10 | 12 | 3m | | | | 9n | | cggac | 2508 | gtagcaacggaccacagaa | 2526 |

The table 17 lists 9 most efficacious antisense reported in the literature. For each of the ASOs listed, the name used in the reported study is indicated, and the beginning and ending points of each sequence corresponding to the study is listed in the last column. The specificity was reflected by different hits under the title of match. “Efficacy” refers to the approximate degree to which gene expression was inhibited in the study. Where only data corresponding to mRNA levels are reported in the indicated study, “BCL2” means B-cell CLL/lymphoma 2 molecule. “VCAM” means vascular cell adhesion molecule. “PKC” means protein kinase C. “p53” means oncogene inhibitor. “TNF” means tumor necrotic factor. “PGY1” means Xenopus kinesin-like protein.

TABLE 17
Nine most efficacious ASO molecules reported in literature.

| | Total | 100% | | 80-95% | | <80% | | Start | | End |
|---|---|---|---|---|---|---|---|---|---|---|
| | Hit | Match | | Match | | Match | Pattern | Point | Sequence | Point |
| BCL-2 | 34 | 9m | 1n | 1n | 1m | 12n | | 33 | tggcgcacgctgggagaac | 51 |

Cotter et al., 1994, Oncogene 9: 3049-3055

| | | | | | | | | |
|---|---|---|---|---|---|---|---|---|
| TNF | 22 | 12m | 3n | 10n | cggga | 582 | agcatgatccgggacgtgg | 600 |

d'Hellencourt et al., 1996, Biochim. Biophys. Acta 1317: 168-174

| | | | | | | | |
|---|---|---|---|---|---|---|---|
| VCAM | 40 | 6m | 8n | 22n | 2866 | aacccagtgctccctttgct | 2847 |

Lee et al., 1995, Shock 4: 1-10

| | | | | | | | | |
|---|---|---|---|---|---|---|---|---|
| P53 | 91 | 30m | 2 | 1n | 59n | 1224 | cctgctcccccctggctcc | 1206 |

Bishop et al., 1996, J. Clin. Oncol. 14: 1320-1326

| | | | | | | | |
|---|---|---|---|---|---|---|---|
| PGY1 | 8 | 3m | 1m | 5n | 428 | ccatcccgacctcgcgct | 411 |

Alahari et al., 1996, Mol. Pharmacol. 50: 808-819

| | | | | | | | | |
|---|---|---|---|---|---|---|---|---|
| RAF | 27 | 5m | 2n | 7n | 13n | 2503 | tcccgcctgtgacatgcatt | 2484 |

Monia et al., 1996, Nature Med. 2: 668-675

| | | | | | | | |
|---|---|---|---|---|---|---|---|
| PKC-a | 18 | 4m | 2n | 12n | 41 | aaaacgtcagccatggtccc | 22 |

Dean et al., 1994, J. Biol. Chem. 269: 16416-16424

| | | | | | | | | |
|---|---|---|---|---|---|---|---|---|
| CD54 | 336 | 8m | 1n | 7n | 320n | 1952 | tgagaggggaagtggtggg | 1970 |

Lee et al., 1995, Shock 4: 1-10

| | | | | | | | | |
|---|---|---|---|---|---|---|---|---|
| BCR | 21 | 18m | 1n | 2n | cgggg | 3203 | gtctccggggctctatgggt | 3222 |

Maran et al. 1998, Blood 92 (11): 4336-4343

After careful observation on the profiles of match in each case, it is clear that more 100% of matches and less incomplete matches confers high efficacy on ASOs. Because it is well known in the art that uridine has nucleotide binding properties analogous to those of thymidine, one of skill in the art will recognize that T may also be U.

Therefore, it has been demonstrated herein that ASOs which are efficacious for inhibiting expression of genes comprising a corresponding RNA molecule may be made by selecting an ASO comprising a nucleotide sequence which is completely homologous to its family member and has minimal similarity to any other family members. Surprisingly, two of these nine sequences contain the cleavage sequence (CGGGA in TNF and CGGGG in BCR) the invention recommended. Taken together, ASOs which are efficacious for inhibiting expression of genes encoding a corresponding RNA molecule may be made by selecting an ASO comprising a nucleotide sequence complementary to a region of the corresponding RNA molecule, wherein the region is shared by most, if not all, members of the same gene family but lest, if not none, members of other gene families. Obviously, the region with the cleavage pattern indicated in the invention is able to meet this standard and can be taken as the basis for predicting an efficacious SDSO.

### EXAMPLE-4

### Prospective Design of SDSOs Which is Efficacious for Inhibiting Over-Expression of Other mRNAs Present in Cells and Tissues of a Patient

For the Treatment of Cancers

There are many gene therapy strategies that have been applied for the treatment of cancer, but their common features are to inhibit the expression of a gene in a cell. The preferred strategic approaches of the present invention are to inhibit oncogene expression, to untie the suppression of tumor suppressor genes, to block key pathways to cause pathogenic growth of a cell, and to reestablish apoptosis system within the cell by the administration of a group of specific DSOs loaded in a gene drug.

In order to meet the goal of the invention, a combination of eight basic active double-stranded oligonucleotides and other agents specific to different cases was developed and integrated into a gene drug for a tumor cell. These 19-25 nt double-stranded oligonucleotides include, but are not limited to, H- and N-Ras, PKC-alpha, CDK-2 and 4, Stat-3 and 5, MDM-2, Telomerase, Methyltransferase, HIF, bFGF and VEGF. The strategic targets are related to the suppression of oncogene, activation of oncogene suppressors, blockage of vessel growth, silence of survival gene, interruption of growth factor pathway, initiation of apoptotic activity, and removal of abnormal methylation. Except for the basic ingredients, the compounds of the invention also include other active agents specific to:

Dermogene HPV (E6), CDKN2A, HDC, N-Ras, BCL-2 and -x1.

Lungene: IGF, b-FGF, K-RAS, Neu, HGF, BCL-2 and -x1.

Hepatogene HuH-7 (Hepatoma-derived Growth Factor), rhoB, c-myc, TR3 orphan receptor, TGF-alpha, N-RAS, and HGF.

Leukogene BCL-6, Bcr-Abl, N-Ras

Lymphogene BCL-2, HIF

Prostogene E2F4, Daxx, HIF

Breastogene BRCA1 and 2, erbB-2, Estrogen receptor, HIF

Braintumogene N-RAS

As mentioned above, Dermogene, Lungene, Hepatogene, Leukogene, Lymphogene, Prostogene, Breastogene and Braintumogene are the names of the gene drugs of the invention. In these gene drugs, there are different active compositions which are some SDSO molecules inhibiting the expression of their cognate mRNA molecules. These SDSO molecules and other assistant composition form different gene drugs for the treatment of different cancers.

For the Treatment of Viruses and Fungi

The therapeutic strategies to virus and fingi used in the invention are to prevent and cure viral infection by amplifying natural anti-virus and anti-fungus system in a human. The dsRNA is an excellent antiviral means existing in most biological bodies. This type of drug genes inhibits the functioning of viral RNAs by interfering with active status of its RNAs. These drugs could be used in aerosol, topical or systematic forms for respiratory, gastrointestinal or systematical viral infections, respectively.

Since dsRNAs often exist in virus-infected cells, their products and themselves can play some important biological roles in host-virus interaction. Generally, dsRNAs and their products can definitely cause the response of host defense system. Recently, it is well known that dsRNA can also lead to a RNA interference through the specific process to cut down long dsRNA into 19-25 nt siRNAs that can inactivate cognate mRNA molecule. In plants, it serves as an antiviral defense, and many plant viruses encode suppressors of silencing. The animal cells may employ the RNA silencing mechanisms as part of a sophisticated network of interconnected pathways for cellular defense, RNA surveillance, and developmental control. Taken together, in order to avoid the uncertain effects of dsRNA on cell physiology, we prefer to use small interference RNAs with 19-25 nt as active ingredients of gene drugs against viruses and fingi.

By the way of example, the 21nt double-stranded oligonucleotides against pol, tat and env were screened and selected as a specific gene drug for AIDS, acquired immunodeficiency syndrome. The active ingredients include, but are not limited to,

AIDSogene: Protease (PROT), polymerase (POL), integrase (INT), gp120 and gp41, transactivating protein (TAT), regulator of expression of virion protein (REV), and viral infectivity factor (VIF)

Many other antiviral and antifungal gene drugs can be designed and developed with the method of the invention. These gene drugs may be used topically for superficial infections and intravenously for systematic disease caused by virus or fungi. The drug genes can be efficiently delivered by using liposomes, lipid dissolvent or other carriers.

While this invention has been disclosed with reference to specific embodiments, those of ordinary skills in the art will be able to readily imagine and produce further embodiments and variances, based on the teachings herein, without undue experimentation. The appended claims are intended to be construed to include all such embodiments and equivalent variations. References cited herein are hereby incorporated by reference.

### BRIEF DESCRIPTION OF THE DRAWINGS

FIG. 1. An endogenous RNAI

The sequence of a human let-7 RNA gene is composed of a line of nucleotides. The blue one stands for the sequence encoding the sense strand of let-7 RNA, while the red is for the antisense strand of let-7 RNA. The green one is related to the change of nucleotides in let-7 RNA gene.

AL158152.18 GI:15212042, Human DNA sequence from clone RP11-2B6 on chromosome 9q22.2-31.1

[image]

FIG. 2. BLAST Multiple Sequence Alignments:

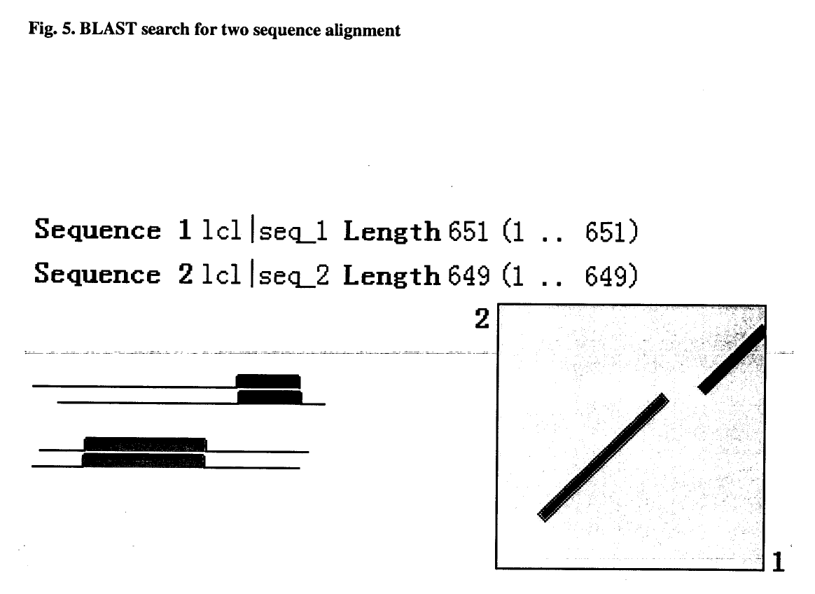

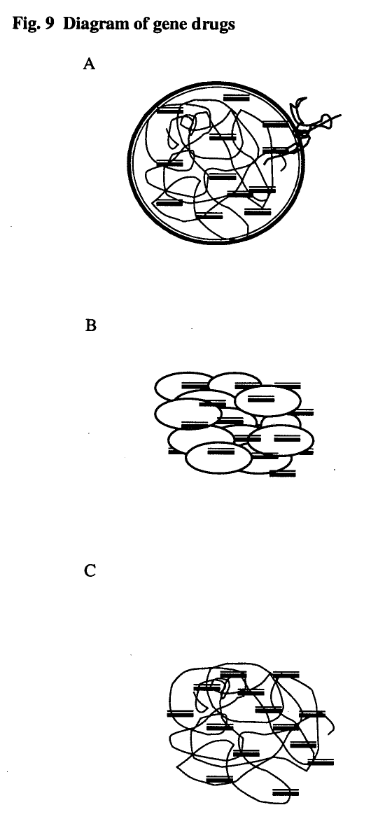

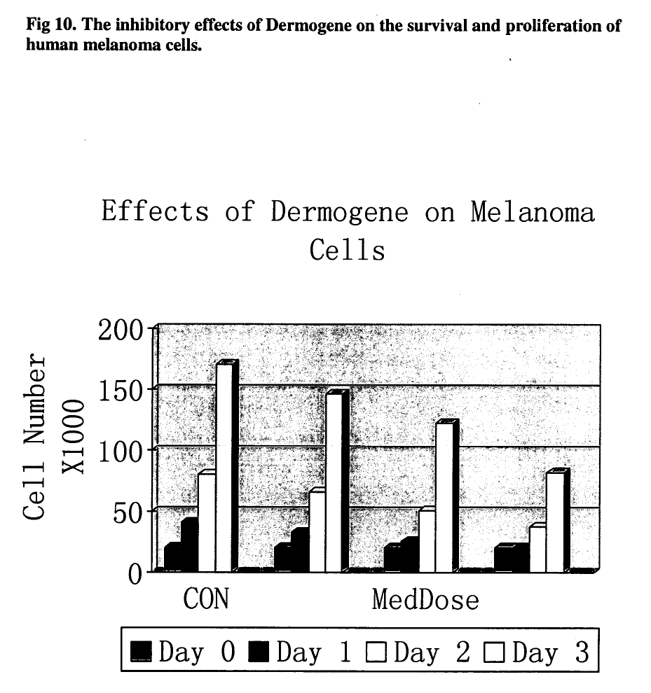

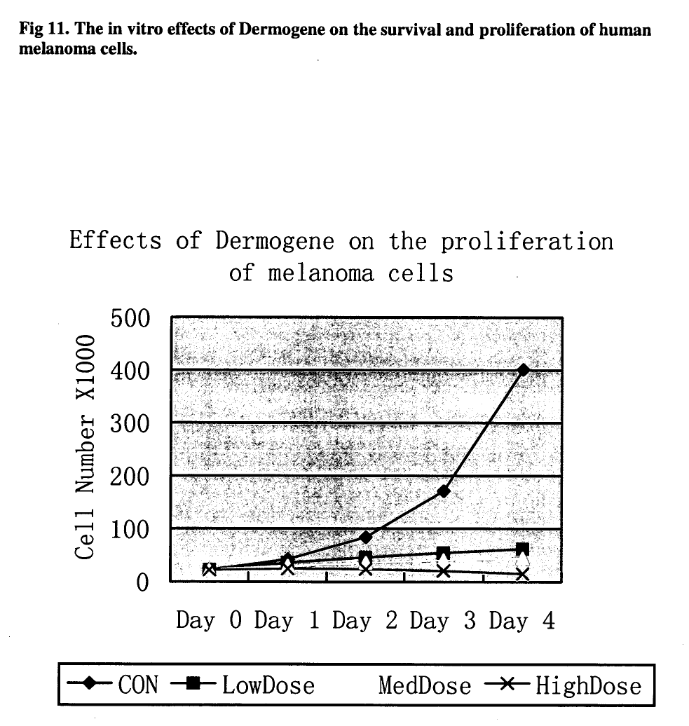

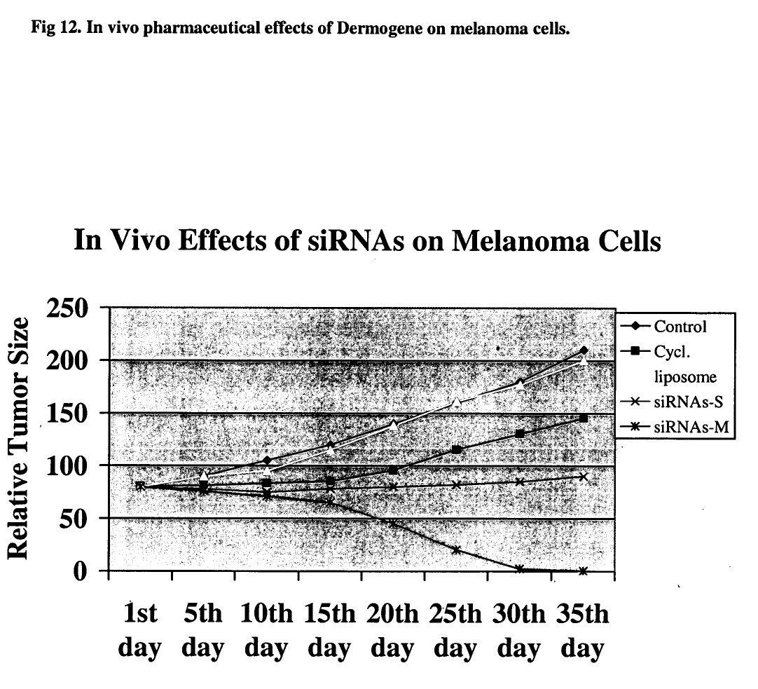

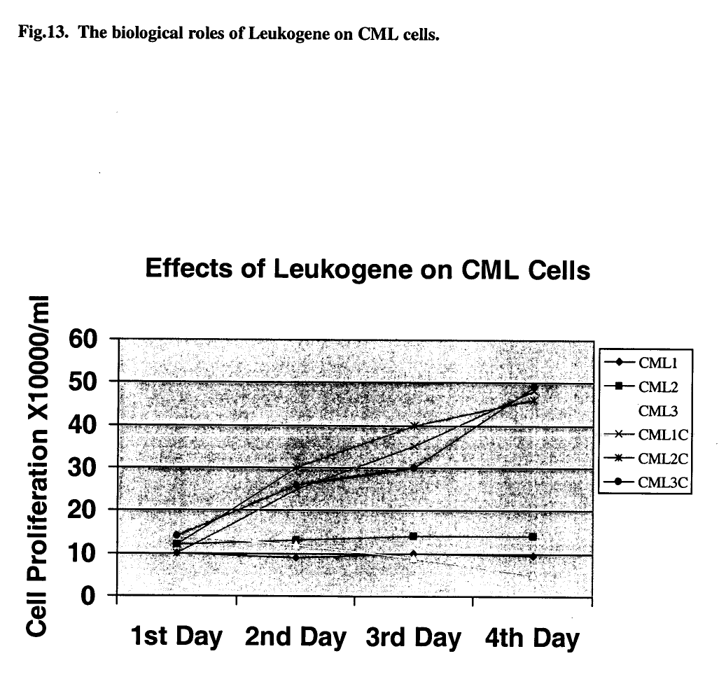

A set of sequences was fished out by a query sequence of human insulin-like growth factor 2 gene.

[image]

| | | Score | E |
|---|---|---|---|
| Sequences producing significant alignments: | | (bits) | Value |
| gi\|32997\|emb\|X07867.1\|HSIGF24B | Human DNA for insulin-like g . . . | 1009 | 0.0 |
| gi\|33003\|emb\|X03562.1\|HSIGF2G | Human gene for insulin-like g . . . | 722 | 0.0 |
| gi\|183100\|gb\|M22373.1\|HUMGFIA2 | Human insulin-like growth fa . . . | 722 | 0.0 |
| gi\|2909374\|emb\|Y16533.1\|OAR16533 | Ovis aries IGF-II gene, ex . . . | 222 | 3e-55 |
| gi\|405977\|gb\|U00665.1\|OAINIGFII4 | Ovis aries insulin-like gr . . . | 208 | 4e-51 |
| gi\|2558855\|gb\|AF020599.1\|ECILGF22 | Equus caballus insulin-li . . . | 198 | 4e-48 |
| gi\|2689877\|gb\|U71085.1\|MMU71085 | Mus musculus insulin-like g . . . | 174 | 5e-41 |
| gi\|15208269\|dbj\|AP003184.1\|AP003184 | Mus musculus genomic DN . . . | 174 | 5e-41 |

FIG. 3. CLUSTAL W (1.81) Multiple Sequence Alignments:

The homologous sequences of human insulin-like growth factor 2 gene derived from different species were aligned and compared with each other by using CLUSTAL W Multiple Sequence Alignments.

| | |
|---|---|
| Sequence format is Pearson | |
| Sequence 1: Ymossambicus | 570 bp |
| Sequence 2: AF79Tilapiamossamb | 549 bp |
| Sequence 3: Y9Oreochromismossa | 387 bp |
| Sequence 4: AF7Gallusgallus | 1066 bp |
| Sequence 5: AJZebrafinch | 564 bp |
| Sequence 6: MMouseinsulin-lik | 543 bp |
| Sequence 7: Rat IGF-2 | 543 bp |
| Sequence 8: human IGF-2 | 543 bp |
| Start of Pairwise alignments | |

[image]

[image]

| | | Score | E |
|---|---|---|---|
| Sequences producing significant alignments: | | (bits) | Value |
| gi\|14773163\|ref\|XM_006402.3\| | Homo sapiens insulin-like grow . . . | 42 | 0.002 |
| gi\|14773161\|ref\|XM_028186.1\| | Homo sapiens insulin-like grow . . . | 42 | 0.002 |
| gi\|14773159\|ref\|XM_028187.1\| | Homo sapiens insulin-like grow . . . | 42 | 0.002 |
| gi\|14773157\|ref\|XM_028184.1\| | Homo sapiens insulin-like grow . . . | 42 | 0.002 |
| gi\|14773155\|ref\|XM_028189.1\| | Homo sapiens insulin-like grow . . . | 42 | 0.002 |

>gi|14773163|ref|XM 006402.3| Homo sapiens insulin-like growth factor 2 (somatomedin A) (IGF2), mRNA Length=1202

Score=42.1 bits (21), Expect=0.002

Identities=21/21 (100%)

Strand=Plus/Plus

| | | | |
|---|---|---|---|
| Query: | 1 | agccgtggcatcgttgaggag | 21 |
| | | \|\|\|\|\|\|\|\|\|\|\|\|\|\|\|\|\|\|\|\|\| | |
| Sbjct: | 544 | agccgtggcatcgttgaggag | 564 |

The specificity of a query sequence selected by systematic selection method was evaluated by Blast search. The results indicated that the total hits were 26, 25 of which are belong to the same gene family, and only one of which is derived from other gene family, suggesting that this query sequence has very high specificity. The experiment indicated that the systematic selection method is a useful and good method even though the process of selection was pretty complicated.

TABLE 4b
gi|33003|emb|X03562.1|HSIGF2G Human gene for insulin-like growth factor II

| | Total | 100% | 80-95% | <80% | | Start | | End |
|---|---|---|---|---|---|---|---|---|
| Seq ID | Hit | Match | Match | Match | Pattern | Point | Sequence | Point |
| 1 | 36 | 25n | | 11n | None | 7534 | agccgtggcatcgttgagg | 7552 |
| 2 | 83 | 25n | 1n | 57n | None | 7543 | atcgttgaggagtgctgtt | 7561 |
| 3 | 84 | 25n | 1n | 58n | None | 7550 | aggagtgctgtttccgcag | 7568 |
| 4 | 65 | 25n | | 40n | None | 7553 | agtgctgtttccgcagctg | 7571 |
| 5 | 42 | 25n | 2n | 15n | None | 7589 | agacgtactgtgctacccc | 7607 |
| 6 | 45 | 25n | | 20n | None | 7591 | acgtactgtgctacccccg | 7609 |
| 7 | 45 | 25n | 1n | 16n | None | 7595 | actgtgctacccccgccaa | 7613 |
| 8 | 51 | 25n | 1n | 25n | None | 7603 | acccccgccaagtccgaga | 7621 |

The table 4b listed other sequences selected by the random selection method. The results showed that all the sequences were not so good as the sequence shown in the FIG. 4, suggesting that the systematic selection method is superior to the random selection method.

FIG. 5. BLAST search for two sequence alignment

This method is useful for selecting homologous sequences with a big gap or different sequence between. After localizing the region of homologous sequence, interested sequence will be selected out as query sequence for further searching and comparing.

[image]

[image]

| | | Score | E |
|---|---|---|---|
| Sequences producing significant alignments: | | (bits) | Value |
| gi\|13702791\|gb\|AC006590.11\|AC006590 | Drosophila melanogaster . . . | 42 | 0.003 |
| gi\|13702790\|gb\|AC008184.4\|AC008184 | Drosophila melanogaster, . . . | 42 | 0.003 |
| gi\|11094921\|gb\|AC084471.1\|AC084471 | Caenorhabditis briggsae . . . | 42 | 0.003 |
| gi\|10799037\|gb\|AF274345.1\|AF274345 | Caenorhabditis elegans l . . . | 42 | 0.003 |
| gi\|7298444\|gb\|AE003659.1\|AE003659 | Drosophila melanogaster g . . . | 42 | 0.003 |
| gi\|15212042\|emb\|AL158152.18\|AL158152 | Human DNA sequence fro . . . | 42 | 0.003 |
| gi\|7211739\|gb\|AF210771.1\|AF210771 | Caenorhabditis briggsae l . . . | 42 | 0.003 |
| gi\|1229025\|emb\|Z70203.1\|CEC05G5 | Caenorhabditis elegans cosm . . . | 42 | 0.003 |
| gi\|4826511\|emb\|AL049853.1\|HS695020B | Human DNA sequence from . . . | 42 | 0.003 |
| gi\|14189751\|dbj\|AP001359.4\|AP001359 | Homo sapiens genomic DN . . . | 42 | 0.003 |

### Alignments

>gi|13702791|gb|AC006590.11|AC006590 Drosophila melanogaster, chromosome 2L, region 36E-, BAC clone BACR13N02, complete sequence

Length=172479

Score=42.1 bits (21), Expect=0.003

Identities=21/21 (100%)

Strand=Plus/Plus

| | | | |
|---|---|---|---|
| Query: | 1 | tgaggtagtaggttgtatagt | 21 |
| | | \|\|\|\|\|\|\|\|\|\|\|\|\|\|\|\|\|\|\|\|\| | |
| Sbjct: | 37997 | tgaggtagtaggttgtatagt | 38017 |

FIG. 7. The cleavage patterns are detected with MUSCA pattern discovery tool. From this gene, most derivative sequences of the cleavage center could be found and used for predicting specific and efficacious sequences. The corresponding results were listed in table 4.

[image]

FIG. 8. Evaluation of an amyloid SDSO designed with the specific cleavage pattern method.

RID: 1000513225-8517-5028

Query=(19 letters)

Database: nt 951,499 sequences; 3,985,165,516 total letters

RID: 1000513225-8517-5028
Query = (19 letters)
Database: nt 951,499 sequences; 3,985,165,516 total letters
>gi|14780094|ref|XM_009710.2| Homo sapiens amyloid beta (A4)
precursor protein (protease
nexin-II, Alzheimer disease) (APP), mRNA Length = 1708

[image]

FIG. 11. displayed that growth-inhibitory effects of Dermogene on cultured human melanoma cells were mediated by the administration of a group of SDSOs every day for four days. For this, 1 ml of melanoma cell suspension in culture medium (2×10⁴/ml) was placed in each well. Cell growth was evaluated on days 0, 1, 2, 3 and 4 by an automatic counter made in Coulter Corporation (n=3). Values given are means±SD expressed as number of cells×10⁴/ml.

[image]

[image]

FIG. 9. displayed that growth-inhibitory effects of Dermogene on cultured human melanoma cells were mediated by the administration of a group of siRNAs for one time. For this, 1 ml of melanoma cell suspension in culture medium (2×10⁴/ml) was placed in each well. Cell growth was evaluated on days 0, 1, 2 and 3 by an automatic counter made in Coulter Corporation (n=3). Values given are means±SD expressed as number of cells×10⁴/ml.

FIG. 11. Effects of injection of cationic liposomes containing Dermogene on the growth of human melanoma transplanted to nude mice. The dark blue line is related to intratumoral injections of PBS (30 ul) every other day. The yellow line means intratumoral injections of empty liposomes (200 nmol liposome in 30 ul) every other day. The light blue line stands for intratumoral injection of liposomes containing Dermogene (5 ug mixture of Dermogene and 200 nmol liposome in 30 ul) every other day. The pink line means intratumoral injection of 30 ul liposomes containing 1 mg Cyclophosphamide. The dark brown line stands for intratumoral injections of liposomes containing Dermogene (5 ug mixture of Dermogene and 200 nmol liposome in 30 ul) and 1 mg Cyclophosphamide every day. Melanoma nodules were evaluated by measuring the size every 5 days with the aid of microcallipers, and tumor volume and relative tumor size were calculated.

FIG. 12. The biological roles of Leukogene on CML cells.

FIG. 12. illustrated the effects of Leukogene in the dose of 100 ng/ml and 200 nmol empty liposome on the proliferation of CML cells derived from (CML1 and CML1C) patient 1, (CML2 and CML2C) patient 2, and (CML3 and CML3C) patient 3. Cell numbers are the average obtained from three wells.

[image]

What is claimed is:

1. A process for the screen, identification or prediction, and assembly of 19-25 nt double-stranded oligonucleotides as active pharmaceutical compositions for the treatment of a variety of viral infection, malignant tumors, and genetic and metabolic diseases, which includes the following steps:
A) screening the disease-causing genes, over-expressing in cells and/or tissues, with the gene-chip and protein-chip microarrays,
B) identifying a specific DNA sequence within the abnormal gene encoding a protein or playing other biological roles with the assistance of computer and specific software,
C) predicting efficacious 19-25 nt double-stranded oligonucleotides with a 5′-AU(T)CCG-3′ or 5′-U(T)CCCG-3′ special pattern complementary to at least a portion of a RNA molecule, and
D) making sure that selected sequence is not localized within the stem-loop of target mRNA with any related software.

2. The process according to claim 1, wherein identifying specific DNA sequences in the human genome includs the steps of:
(a) identifying endogenous short interfering RNA (siRNA) sequences in the human genome with the assistance of computer and specific software,
(b) searching candidate sequence with conserved patterns from the same gene family in different species by using multiple sequence alignment and pattern discovery algorithm as well as Blast searches of Genebank,
(c) selecting a specific DNA sequence with the length of 19-25 nucleotides which is 100% homologous to most, if not all, members of this gene family in human genomic databases,
(d) valuating the specific 19-25 nt sequence by the standard in which there is minimal similarity to any other gene families and 95-100% homologous to any members of the same human gene family through Blast Alignment of Genebank.

3. The process according to one of claims 2, wherein the special pattern such as 5′-CGGAU-3′ is a critical portion of a specific 19-25 nt sequence, which is the base for selecting a region in a given genomic RNA as both a target and a drug.

4. The process according to claim 1, 2 or 3, wherein the 19-25 nt double-stranded oligonucleotides may be a 19-25 nt dsRNA, a 19-25 nt sRNA-cDNA, or a 19-25 nt dsDNA.

5. The process according to claim 4, wherein the cDNA in said sRNA-cDNA is an antisense oligonucleotide, while sRNA is related to a sense oligonucleotide.

6. The process according to claim 1, 2, 3 and 4, wherein the 19-25 nt double-stranded oligonucleotides can specifically hybridize with at least a 19-nucleobase portion of an active site on a nucleic acid molecule encoding a protein or playing other functions, and interfere with or shut off target RNAs, and/or regulate the DNA methylation of corresponding regions of genome derived from human and other species.

7. The process according to claim 2, wherein said endogenous RNAi is a sequence occurring in an intergenetic area or an intron region, where a 19-25 nt stem-loop structures can be identified.

8. The process according to claim 6, wherein target RNAs include mRNA or other types of RNA molecules.

9. Pharmaceutical compositions of gene drugs such as Dermogene, Lungene, Hepatogene, Leukogene, Lymphogene, Prostogene, Breastogene Braintumogene and Skin-whitogene including but being not limited to part or all of the following components:
single or a group of specific 19-25 nt dsRNA, 19-25 nt sRNA-cDNA, 19-25 nt dsDNA and/or single-stranded RNA and/or DNA with the special pattern, 5′-CGGAT(U)-3′ or its derivative sequences,
one or more nucleic acid condensation agents, or none,
one or more pharmaceutically acceptable carriers,
one or more specific cell-targeting proteins, and
other active agents and additional materials.

10. A pharmaceutical composition according to claim 9, wherein the 19-25 nt double-stranded oligonucleotides with the special pattern such as 5′-CGGAU-3′ or other 5′-CGGNN-3′ can efficiently inhibit expression of a gene in an animal, especially a human.

11. A pharmaceutical composition according to claim 9 wherein a group of oligonucleotides are more than one double-stranded oligonucleotides, each of which is complementary to the specific target sequence within a given RNA.

12. The compositions of gene drugs according to claim 1 and 9, wherein the double-stranded oligonucleotides have a cleavage pattern comprising SEQ ID #: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51.

13. The compound of gene drugs according to claim 1 and 9, wherein the mixture comprises at least one double-stranded oligonucleotide molecule, or different double-stranded oligonucleotides, different dose of the same agent, or any combination thereof.

14. The compound of claim 1, 9 and 13, wherein the double-stranded oligonucleotides can contain at least one special pattern which can be localized in any place in an oligonucleotide sequence.

15. The compound of claim 1, 9, 13 and 14, wherein the special pattern in the antisense strand of SDSO or antisense oligonucleotide (ASO) molecule includes but be not limited to AU(T)CCG, U(T)U(T)CCG, GU(T)CCG, CU(T)CCG, GCCCG, U(T)CCCG, ACCCG, CCCCG, AACCG, U(T)ACCG, GACCG, CACCG, AGCCG, GGCCG, CGCCG, and U(T)GCCG in the order of 5′ to 3′.

16. The compound of claim 1, 9, 13 and 14, wherein the double-stranded oligonucleotides can be a chimeric oligonucleotides.

17. A composition comprising the compound of claim 9 and a pharmaceutically acceptable carrier or diluents.

18. The composition of claim 9 and 17 further comprising a colloidal dispersion system.

19. A simplified method for predicting and selecting a specific and efficacious SDSO or antisense oligonucleotide (ASO) molecules, which includes the identification of a special pattern which can be localized in any position of an oligonucleotide sequence and the evaluation of the specificity of a selected sequence.

20. A composition comprising of the compound of gene drugs such as Dermogene, Lungene, Hepatogene, Leukogene, Lymphogene, Prostogene, Breastogene and Braintumogene as well as cosmetics such as Skin-whitogene.
